(19) United States
(12) Patent Application Publication
(10) Pub. No.: US 2008/0261891 A1
(43) Pub. Date: Oct. 23, 2008

(54) COMPOSITIONS AND METHODS FOR USING SYRINGOPEPTIN 25A AND RHAMNOLIPIDS

(75) Inventor: Bart C. Weimer, Logan, UT (US)

Correspondence Address: UTAH STATE UNIVERSITY/WORKMAN NYDEGGER, 570 RESEARCH PARK WAY, SUITE 101, NORTH LOGAN, UT, 84341, US

(73) Assignee: Utah State University, North Logan, UT (US)

(21) Appl. No.: 12/030,726

(22) Filed: Feb. 13, 2008

Related U.S. Application Data
(60) Provisional application No. 60/890,117, filed on Feb. 15, 2007.

(51) Int. Cl. A61K 38/00; A61P 43/00

(52) U.S. Cl. 514/13; 514/2

(57) ABSTRACT

The present invention provides a therapeutic composition having at least one syringopeptin and at least one rhamnolipid so that the composition has one or more of the following activities: antibacterial; antifungal; and antitumor activity. The therapeutic composition includes the following: a therapeutically effective amount of a syringopeptin; a therapeutically effective amount of a rhamnolipid; and a pharmaceutically acceptable carrier. Additionally, the present invention provides a method for inhibiting or treating cancer or a microbial infection in a subject, wherein the method includes the following: providing a subject in need of inhibition or treatment of cancer or a microbial infection; and administering a therapeutic amount of a therapeutic composition to the subject so as to inhibit or treat the cancer or microbial infection.

### CROSS-REFERENCE TO RELATED APPLICATIONS

This application claims the benefit of U.S. Provisional Application No. 60/890,117, filed Feb. 15, 2007 entitled “COMPOSITIONS AND METHODS FOR USING SYRINGOPEPTIN 25A AND RHAMNOLIPIDS” which application is all hereby incorporated by reference herein in their entireties, including but not limited to those portions that specifically appear hereinafter, the incorporation by reference being made with the following exception: In the event that any portion of the above-referenced application is inconsistent with this application, this application supercedes said above-referenced application.

### FIELD OF THE INVENTION

This invention generally relates to compounds having therapeutic properties. More particularly, the present invention relates to a composition having syringopeptin 25A and rhamnolipids for use as an antimicrobial, antitumor or for other prophylactic or therapeutic treatments.

### BACKGROUND OF THE INVENTION

Very few developments in the history of science have had such a profound impact upon human life as advances in controlling pathogenic organisms. It was not until the late 19^{th} and early 20^{th} century that the work of Pasteur and Koch established microorganisms as the cause of infectious diseases and provided strategies that led to rational prevention and control strategies. Though the application of antimicrobial agents preceded the understanding of their action, the first group of compounds discovered to suppress bacterial infections were sulphonamides. The success of sulphonamides stimulated a massive hunt for more effective antimicrobial compounds. Florey and Chain succeeded in isolating an impure but highly active preparation of penicillin, publishing their results in 1940. The enormous success of penicillin quickly diverted a great deal of scientific effort towards the search for other antibiotics leading to the discovery of approximately 3,000 named antibiotics. Of these, 50 have met with clinical use, but even fewer are commonly used in treating microbial diseases.

The initial effectiveness of the antibiotics against bacterial infections has been partly overcome by the emergence of resistant strains of bacteria. Antibiotic resistance is a difficult problem to overcome because of the accelerated evolutionary adaptability of the microbes, overuse of antibiotics, and the lack of patients completing prescribed dosages. Curable diseases such as gonorrhea and typhoid are becoming difficult to treat due to resistance issues. Bacteria resistant to vancomycin, one of the last broadly effective antibiotics, are becoming increasingly prevalent in hospitals.

In order to keep infectious agents at bay, new antimicrobial compounds must constantly be developed. In order to develop and discover drugs effective against bacteria, we must first understand the mechanisms of antibiotic resistance. Advances in genomics allow researchers to more quickly identify biochemical pathways that are susceptible to inhibition or modification. The knowledge obtained from a system-wide genome analysis helps to design effective molecules to inhibit microbes through various pathways by inhibiting multiple targets.

Antibiotics are comprised of a varied group of compounds having little overall structurally and functionally in common except for their antimicrobial activity. Therefore, it is not surprising that they prevent the growth of susceptible bacteria through manifold different molecular mechanisms. For example, antibiotics such as penicillin, cephalosporin, cycloserine, and vancomycin block integral steps in cell wall synthesis. These antibiotics interfere with the biosynthesis of peptidoglycan and damage its cross-linked macromolecular structure leading to arrested growth, eventually killing the microbe.

Antibiotics may also kill microbes by permeabilizing their cell membranes. Representative antibiotics that permeabilize cell membranes can include polymyxin, tyrocidin, and valinomycin. These antibiotics interact with the components of the cell membrane and induce a lesion in the cell membrane. The formation of a lesion in the cell membrane impairs its ability to act as a semi-permeable barrier between the cell and its environment. This causes the cell components to leak from within the cell to outside of the cell and results in the death of the microbe.

Another possible mode of action for antibiotic compounds is through the inhibition of nucleic acid function. Representative antibiotics that inhibit nucleic acid function include rifampicin, actinomycin D, and acridines. These compounds interfere at various stages of nucleotide biosynthesis and the polymerization of nucleotides. The resulting failure to express genes causes the death of the cell.

Antibiotics such as streptomycin, tetracycline, and chloramphenicol work by inhibiting protein synthesis. These compounds bind the subunits of ribosomes and distort the ribosomes enough to prevent a normal codon and anticodon interaction which leads to either inhibition of protein synthesis or synthesis of faulty proteins.

Antibiotics may also work by inhibiting cellular metabolism. For example, sulphonamides inhibit the synthesis of folic acid by competing with p-amino benzoic acid as a substrate for the enzyme tetrahydropteroic acid synthetase.

Even with all of the aforementioned varied mechanisms of action of existing antibacterial compounds, microbial strains that are resistant to all of these antibiotics are becoming an increasingly common phenomenon. In general, gram-negative bacteria are more resistant to antibiotics than are gram-positive bacteria. The increased resistance of gram-negative bacteria to antimicrobial agents may be due to the non-specific permeability barrier presented by the outer membrane. This barrier in gram-negative bacteria might prevent access of the antibiotic molecules to their active site. Gram-positive bacteria do not have this additional non-specific permeability barrier.

Resistance of bacteria to certain antibacterials may be due to some bacteria possessing various defense mechanisms against antibiotics. Examples of inherent defense mechanisms to antibacterials include increasing the translation of antibiotic degrading enzymes and upregulating various antibiotic efflux mechanisms. The simplest form of antibiotic resistance is for the microbe to simply altogether lack the antibacterial target.

There are many biochemical mechanisms that bacteria use to obtain antibiotic resistance. Drug resistance may occur when there is conversion of an active drug to an inactive derivative such as the inactivation of β-lactam antibiotics by β-lactamases. β-lactamases are bacterial enzymes that evolved to break the lactam ring of the β-lactam antibiotics and thus render the antibiotic unable to inhibit cell wall biosynthesis.

Antibiotic resistance may occur when there is an enhancement of alternative metabolic pathways. For example, resistance to antibiotic compounds that inhibit nucleic acid biosynthesis may occur when the pathways responsible for the salvage of purine and pyrimidine bases from nucleic acid catabolism are enhanced. This up-regulation allows for the use of these catabolic products to synthesize new nucleic acids.

Bacteria may become resistant to antibacterial compounds through the synthesis of an additional permeability barrier at the cell membrane. This additional permeability barrier can prevent passive transport as well as other more specific transport mechanisms of antibacterial compounds through the cell membrane. Thus, the antibacterial compounds never reach their targets within the cell.

Bacteria may obtain resistance to certain antimicrobial compounds through a physical modification of the drug-sensitive site. The physical alteration of a protein is due to a change in the nucleotide sequence of the gene that codes for the RNA that is used as a transcript for the translation of the protein. Through billions of rounds of evolutionary pressure, mutations may occur in a particular gene encoding for the protein that is the antibiotic target. If there is a slight change at the active site of the protein, or wherever the antibiotic compound binds, the antibiotic will be ineffective because it will no longer be able to bind. This can happen through the simple swapping of one amino acid for another, through a deletion of an amino acid, or through the addition of an amino acid, preventing the antibiotic from binding with its protein target. For example, resistance to erythromycin in several bacterial species depends on an alteration in a part of a protein of the 50S ribosome subunit that leads to a reduced affinity of ribosomes for binding of the antibiotic erythromycin.

Active efflux of an antibiotic from the cytoplasm is another mechanism that bacteria may use in order to achieve resistance to antibacterial compounds. For example, resistance to tetracycline in several gram-positive as well as gram-negative bacteria depends upon an ATP dependent efflux system present in the cellular membrane.

Overuse of antibiotics is thought to be the most important factor contributing to bacteria gaining antibiotic resistance. The unregulated and often unnecessary exposure of different bacteria to antibiotics increases the chances that a resistant strain of bacteria may arise. If the bacteria were never exposed to the antibiotic, they wouldn't have a chance to adapt to the antibiotic and become resistant. By unnecessarily exposing the bacteria to antibiotics, mankind is effectively running a large experiment where we are selecting for resistant strains. Mechanisms by which microbes gain resistance may be through spontaneous mutations, transduction, transposition or conjugation. Once the resistant strains of bacteria are established, they may spread with impunity.

Development of multi-drug antibiotic resistance in bacteria is one of the most urgent issues facing the health sciences today. Unfortunately, antibiotic resistance is an increasingly growing health problem throughout the world. With every new antimicrobial compound discovered or synthesized, our technology is only a step ahead of the microbes, who soon find a way to gain resistance to the antibiotic. The few remaining and broadly effective antibiotics, coupled with the increasing resistance to the available antibiotics have created a desperate effort to discover new antibiotic compounds. There is no certain way to circumvent the microbes from developing new ways to defeat the effect of antibacterial compounds. New compounds that inhibit microbes through different mechanisms need to be continuously developed.

Additionally, many compositions with antimicrobial properties have been found to be useful in other applications. In part, this is because the activity imparted by the antimicrobial can also have effects against other maladies. As such, some antibiotics have found uses in other fields of treatment. For example, siomycin A was originally used as an antimicrobial, but when it was found to inhibit a gene involved in cell proliferation, it became useful for inhibiting the uncontrolled proliferation of cancerous cells. In another example, rapamycin, which was originally used as an antibiotic is now also used to help reduce the rejection of transplanted organs and to prevent restinosis. Therefore, it may be beneficial to study antibiotic compositions to determine whether or not they can be used to prevent, inhibit, or treat other maladies.

### SUMMARY OF THE INVENTION

Generally, the present invention includes a therapeutic composition that can be useful for inhibiting or treating a malady, such as cancer, microbial infection, or tuberculosis. The therapeutic composition may comprise a therapeutically effective amount of a syringopeptin and a therapeutically effective amount of a rhamnolipid in a pharmaceutically acceptable carrier. In particular, the syringopeptins of the therapeutic composition have 22 or 25 amino acids.

In one embodiment, the syringopeptin has a polypeptide sequence as in SEQ ID No. 1 or 2. In one embodiment, the N-terminal amino acid residue of the syringopeptin is acylated by a 3-hydroxylated fatty acid chain comprising 10 or 12 carbon atoms. In one embodiment, the C-terminal amino acid carboxyl group of the syringopeptin is linked to an amino acid residue 7 residues away and form an 8-membered lactone macrocycle.

In one embodiment, the rhamnolipid has a structure as in Formula I as follows:

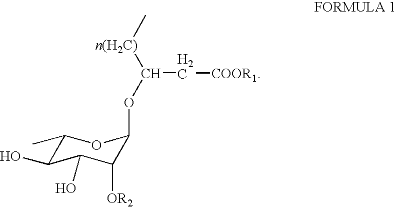

In accordance with Formula 1, the rhamnolipid can be characterized as follows: n is from 4-12; R₁ is H or 3-hydroxydecanoate; and R₂ is L-rhamnosyl or H. Optionally, the ratio of syringopeptin and rhamnolipid ranges from about 1:10 to about 10:1.

In one embodiment, the therapeutically effective amounts of syringopeptin and rhamnolipid achieve a minimum inhibitory concentration in a subject sufficient to prevent, alleviate, or eliminate a microbial infection, such as tuberculosis. Alternatively, the therapeutically effective amounts of syringopeptin and rhamnolipid achieve a minimum inhibitory concentration in a subject sufficient to prevent tumor formation, reduce tumor growth, reduce tumor size, or kill tumor cells.

In one embodiment, the present invention provides a method for inhibiting or treating a microbial infection in a subject. Such a method can include providing a subject in need of inhibition or treatment for a microbial infection, and administering a therapeutic amount of the therapeutic composition to the subject so as to inhibit or treat the microbial infection. As such, the method may achieve a minimum inhibitory concentration in a subject that is sufficient to prevent, alleviate or eliminate a microbial infection. For example, the microbial infection that may be treated could be tuberculosis.

In one embodiment, the present invention provides a method for inhibiting or treating cancer in a subject. Such a method can include providing a subject in need of inhibition or treatment of cancer, and administering a therapeutic amount of the therapeutic composition to the subject so as to inhibit or treat the cancer. As such, the method may achieve a minimum inhibitory concentration in a subject that is sufficient to prevent, alleviate or eliminate a neoplasia condition

In one embodiment, the compositions of rhamnolipids and syringopeptins of the present invention may also be combined with a suitable member from the family of Pseudomonadaceae for the purposes of creating an effective herbicide.

### BRIEF DESCRIPTION OF THE DRAWINGS AND TABLES

To further clarify the above and other advantages and features of the present invention, a more particular description of the invention will be rendered by reference to specific embodiments thereof which are illustrated in the appended drawings and described in the appended tables. It is appreciated that these drawings depict only typical embodiments of the invention and are therefore not to be considered limiting of its scope. The invention will be described and explained with additional specificity and detail through the use of the accompanying drawings.

### I. FIGURES

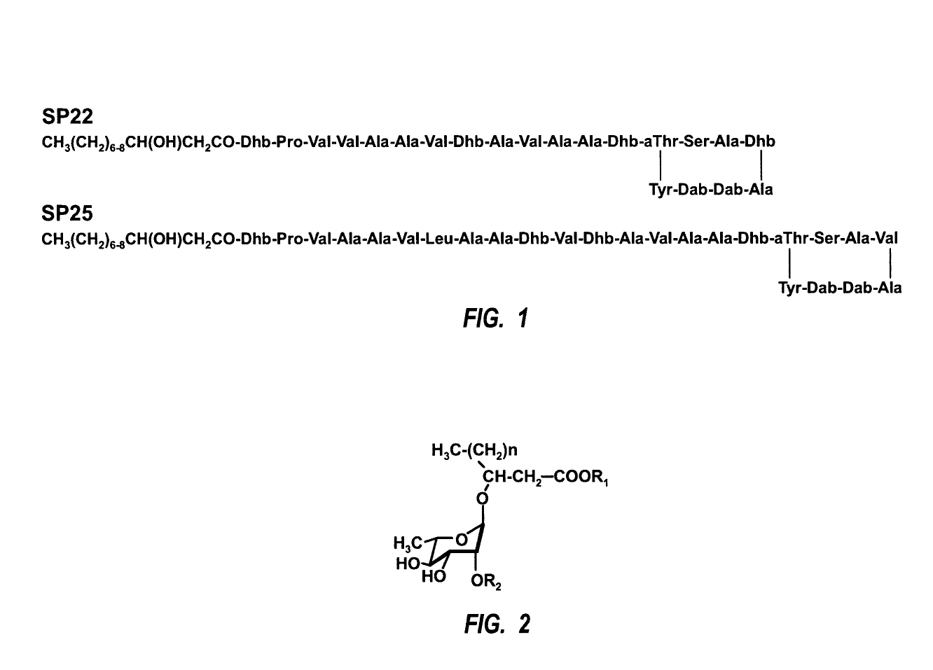

FIG. 1 is a schematic diagram of the structures of two embodiments of syringopeptins with amino acid backbones of 22 and 25 residues.

FIG. 2 is a schematic diagram of a general structure of an embodiment of a rhamnolipid.

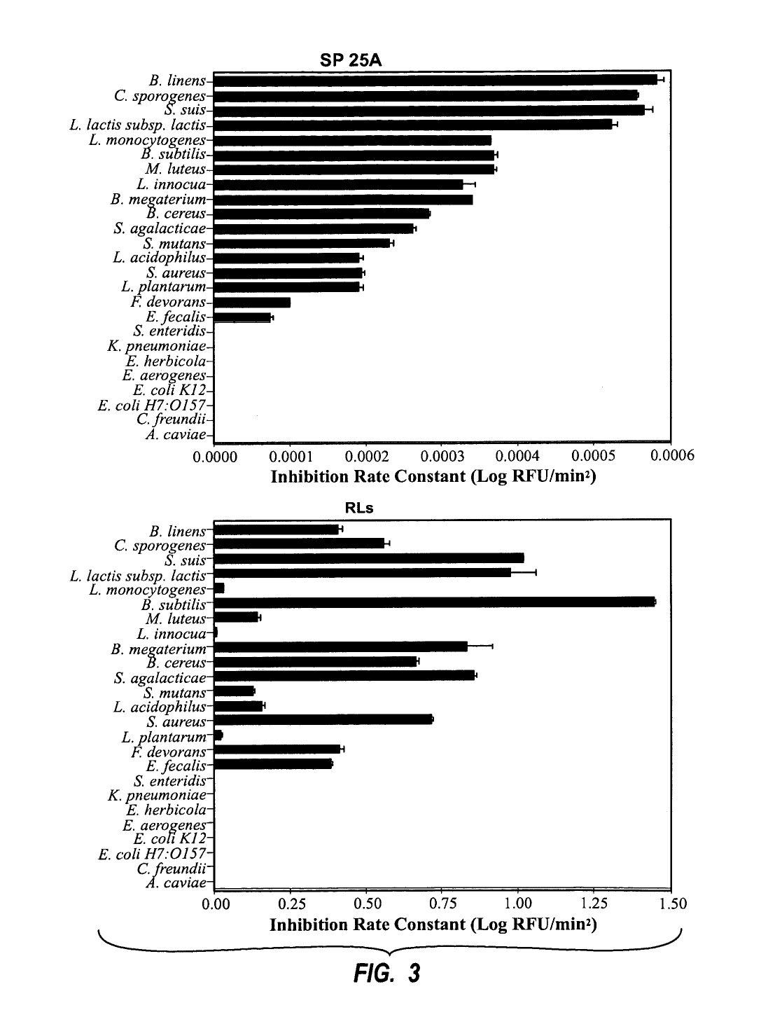

FIG. 3 includes graphs that illustrate the inhibition rate for syringopeptins and rhamnolipids.

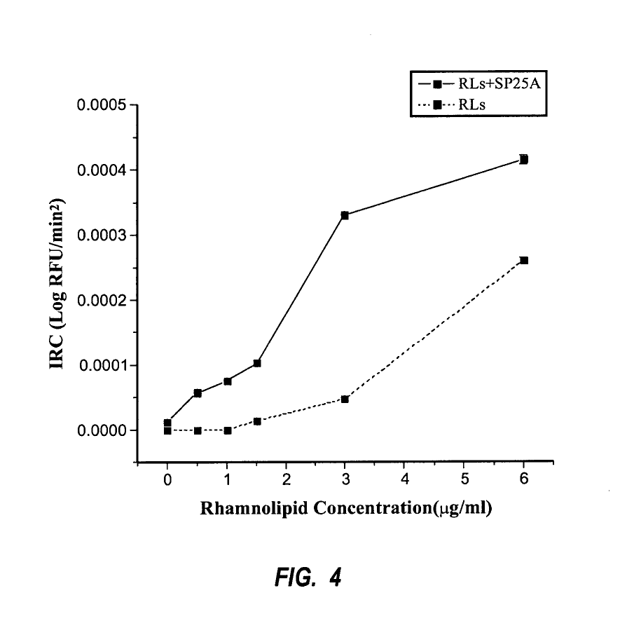

FIG. 4 includes a graph that illustrates the inhibition rate for L. monocytogenes with RLs alone, and with rhamnolipids combined with SP 25A.

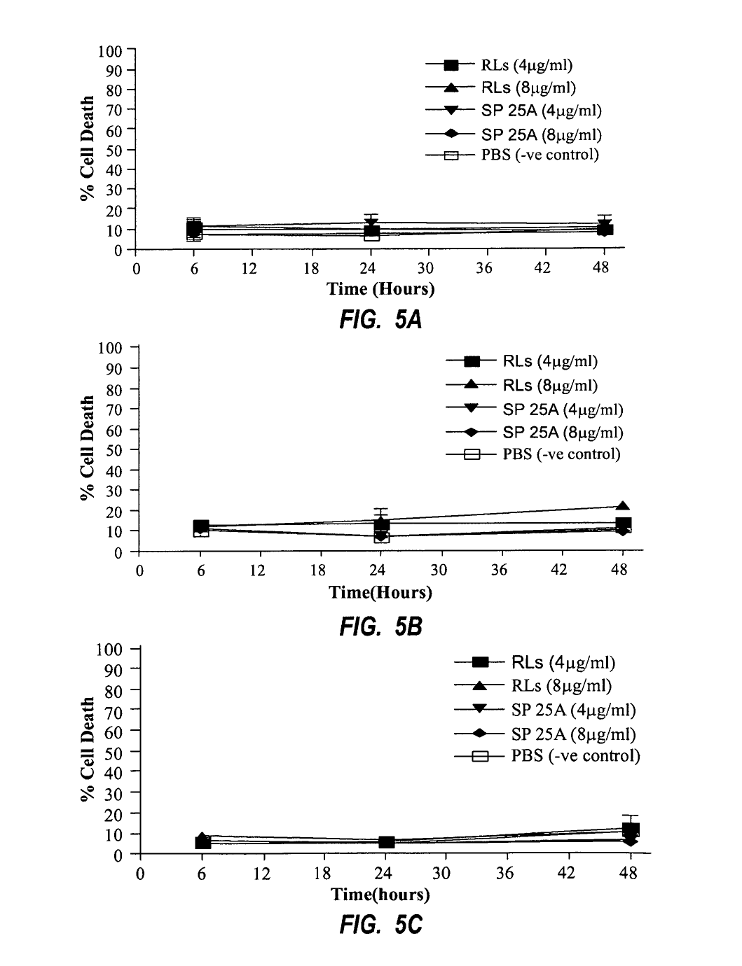

FIG. 5 includes graphs that illustrate the toxicity of RLs and SP 25A against cell cultures (e.g., STC (panel A), HEK 293 (panel B) and LL-47 (panel C)).

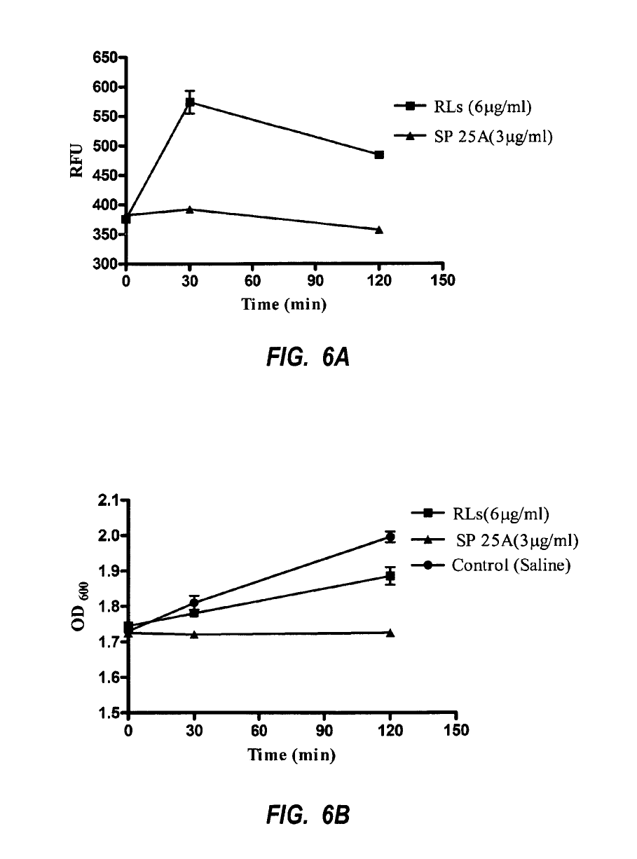

FIG. 6A includes a graph that illustrates the amount of membrane permeabilization in response to SP 25A and RLs.

FIG. 6B includes a graph that illustrates the amount of cell growth in response to being treated with SP 25A, RLs and saline (control) over a period of 120 min

### II. TABLES

Table 1 includes a list of microorganisms that can be used for antimicrobial screening and their growth conditions.

Table 2 includes a list of microorganisms and the MICs and IRs of SP 25A and RLs.

Table 3 includes a list of functional categories that contained the genes that were significantly differentially expressed in response to treatment with sub-MIC doses of RLs.

Table 4 includes a list of significantly differentially regulated genes during exposure to RLs.

Table 5 includes a list of functional categories that contained the genes that were significantly differentially expressed in response to treatment with sub-MIC doses of SP 25A.

Table 6 includes a list of significantly differentially regulated genes during exposure to SP 25A.

Table 7 includes a list of a therapeutic solutions of RLs and SP 25A.

Table 8 includes a list of therapeutic compositions of RLs and SP 25A.

Table 9 includes effects of SP25A on selected human cancer cell lines as % cytotoxicity.

### DETAILED DESCRIPTION

The therapeutic compositions of the present invention may comprise a therapeutically effective amount of syringopeptin, combined with a therapeutically effective amount of rhamnolipids in a pharmaceutically acceptable composition. In particular, the syringopeptins of the therapeutic composition have 22 or 25 amino acids.

This invention is directed to compositions possessing one or more of the following activities: antibacterial, antifungal and antitumor activity. However, prior to describing this invention in further detail, the following terms will first be defined:

As used herein, the term “SP” or “syringopeptin” is meant to refer to a class of cyclic peptides substituted with fatty acids. As shown in FIG. 1, SP 25A contains 25 amino acids and SP 22 contains 22 amino acids.

As used herein, the term “RL” or “rhamnolipids” is meant to refer to a class of biosurfactants that consist of one or more moieties of a rhamnose sugar covalently linked to a hydroxy acid, having a variable length of the carbon atom chain. FIG. 2 shows an embodiment of a general rhamnolipid.

As used herein, the term “MIC” or “minimum inhibitory concentration” is meant to refer to the lowest concentration of a compound that measurably inhibits growth.

As used herein, the term “IR” or “inhibitory rate” is meant to refer to the rate at which a compound inhibits growth.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation. Neoplastic cells can be malignant or benign.

As used herein, the terms “antineoplastic agent”, “antineoplastic chemotherapeutic agent”, “chemotherapeutic agent”, “antineoplastic” and “chemotherapeutic” are used interchangeably herein and refer to chemical compounds or drugs which are used in the treatment of cancer e.g., to kill cancer cells and/or lessen the spread of the disease.

As used herein, the term “herbicide” or “herbicidal” refers to materials which destroy or inhibit plant growth, such as by desiccation or defoliation, for example, to act as a harvest aid or to control weed growth.

As used herein, the term “component” is meant to refer to any substance or compound. A component can be active or inactive.

As used herein, the term “active component” is meant to refer to a substance or compound that imparts a primary utility to a composition or formulation when the composition or formulation is used for its intended purpose. Examples of active components include pharmaceuticals, dietary supplements, alternative medicines, and nutraceuticals.

As used herein, the term “inactive component” is meant to refer to a component that is useful or potentially useful to serve in a composition or formulation for administration of an active component, but does not significantly share in the active properties of the active component or give rise to the primary utility for the composition or formulation. Examples of suitable inactive components include, but are not limited to, enhancers, excipients, carriers, solvents, diluents, stabilizers, additives, adhesives, and combinations thereof.

As used herein, the term “excipient” is meant to refer to the inactive substances used to formulate pharmaceuticals as a result of processing or manufacture or used by those of skill in the art to formulate pharmaceuticals, dietary supplements, alternative medicines, and nutraceuticals for administration to animals or humans. Preferably, excipients are approved for or considered to be safe for human and animal administration.

As used herein, the term “pharmaceutical” is meant to refer to any substance or compound that has a therapeutic, disease preventive, diagnostic, or prophylactic effect when administered to an animal or a human. The term pharmaceutical includes prescription drugs and over the counter drugs. Pharmaceuticals suitable for use in the invention include all those known or to be developed.

As used herein, the term “alkyl” means a linear or branched saturated monovalent hydrocarbon radical of one to ten carbon atoms, preferably one to six carbon atoms, e.g., methyl, ethyl, propyl, 2-propyl, n-butyl, iso-butyl, tert-butyl, pentyl, and the like.

As used herein, the term “cycloalkyl” means a saturated monovalent cyclic hydrocarbon radical of three to six ring carbons, e.g., cyclopropyl, cyclopentyl, cyclohexyl, and the like.

As used herein, the term “halo” means fluoro, chloro, bromo, or iodo, preferably fluoro and chloro.

As used herein, the term “optional” or “optionally” means that the subsequently described event or circumstance may but need not occur, and that the description includes instances where the event or circumstance occurs and instances in which it does not.

As used herein, “amino acid” refers to any of the naturally occurring amino acids, as well as synthetic analogs (e.g., D-stereoisomers of the naturally occurring amino acids, such as D-threonine) and derivatives thereof. Alpha-amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (ed.), Synthesis of Optically Active.alpha.-Amino Acids, Pergamon Press (1989); Evans et al., J. Amer. Chem. Soc., 112:4011-4030 (1990); Pu et al., J. Amer. Chem. Soc., 56:1280-1283 (1991); Williams et al., J. Amer. Chem. Soc., 113:9276-9286 (1991); and all references cited therein. The present invention includes the side chains of unnatural amino acids as well.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4^{th} edition J. March, John Wiley and Sons, New York, 1992).

As used herein, the term “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

As used herein, the term “pharmaceutically acceptable acid addition salts” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Groups which form pharmaceutically acceptable acid addition salts include amines, hydrazines, amidines, guanidines, substituted aryl/heteroaryl and substituted alkyl groups that carry at least a nitrogen bearing substitutent such as amino, uanidine, amidino, uanidine and the like.

Amine groups are represented by the formula —NR′R″ where R′ and R″ are independently hydrogen, alkyl, substituted alkyl, alkenyl, substituted alkenyl, aryl, substituted aryl, cycloalkyl, substituted cycloalkyl, heterocyclic, heteroaryl, substituted heteroaryl, and where R′ and R″, together with the nitrogen to which they are attached, form a heterocyclic or heteroaryl group.

As used herein, the term “treating” or “treatment” of a disease includes: (a) preventing the disease, i.e. causing the clinical symptoms of the disease not to develop in a mammal that may be exposed to or predisposed to the disease but does not yet experience or display symptoms of the disease; (b) inhibiting the disease, i.e., arresting or reducing the development of the disease or its clinical symptoms; or (c) relieving the disease, i.e., causing regression of the disease or its clinical symptoms.

As used herein, the term “unit dosage form”, as used herein, refers to physically discrete units suitable as unitary dosages for human and animal subjects, each unit containing a predetermined quantity of pharmacological agent calculated in an amount sufficient to produce the desired effect in association with a pharmaceutically acceptable diluent, carrier or vehicle.

As used herein, the term “anti-fungal” or “anti-bacterial” means that growth of the fungus or bacterial is inhibited or stopped.

As used herein, the term “anti-tumor” means the compound has the property of inhibiting the growth of tumor cells.

As used herein, the term “bacteriostatic” means the compound has the property of inhibiting bacterial or fungal multiplication, wherein multiplication resumes upon removal of the active compound. For a bacteriostatic compound, its minimum bacteriocidal concentration (“MBC”) is defined as being greater than 4 times its minimum inhibitory concentration.

As used herein, the term “bacteriocidal” or “fungicidal” means that the compound has the property of killing bacteria or fungi. Bacteriocidal/fungicidal action differs from bacteriostasis or fungistasis only in being irreversible. For example, the “killed” organism can no longer reproduce, even after being removed form contact with the active compound. In some cases, the active compound causes lysis of the bacterial or fungal cell; in other cases the bacterial or fungal cell remains intact and may continue to be metabolically active. A bacteriocidal compound exhibits a MBC that is less than 4 times its MIC. Similarly, a fungicidal compound exhibits a minimum fungicidal concentration (“MFC”) that is defined as being less than 4 times its MIC.

As used herein, the term “cancer” may include, but is not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors and germ cell tumors; thyroid cancer including thyroid adenocarcinoma and medullar carcinoma; and renal cancer including adenocarcinoma and Wilms' tumor.

As used herein, the term “cancer treatment” may include, but is not limited to, chemotherapy, radiotherapy, adjuvant therapy, or any combination of the aforementioned methods. Aspects of treatment that may vary include, but are not limited to dosages, timing of administration or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment for a patient.

As used herein, an “agent for prevention of cancer or tumorigenesis” refers to any agent able to counteract any process associated with cancer or tumorigenesis.

As used herein, a “subject” or a “patient” refers to any mammal (preferably, a human), and preferably a mammal that may be susceptible to tumorigenesis or cancer associated with the aberrant expression of a gene or genes. Examples include a human, a non-human primate, a cow, a horse, a pig, a sheep, a goat, a dog, a cat or a rodent such as a mouse, a rat, a hamster, or a guinea pig. Generally, the invention is directed toward use with humans.

As used herein, the term “sample” is any cell, body tissue, or body fluid sample obtained from a subject. Body fluids include, for example, lymph, saliva, blood, urine, and the like. Samples of tissue and/or cells for use in the various methods described herein can be obtained through standard methods including, but not limited to, tissue biopsy, including punch biopsy and cell scraping, needle biopsy; or collection of blood or other bodily fluids by aspiration or other suitable methods.

As used herein, the terms “an effective amount”, “therapeutic effective amount”, or “therapeutically effective amount” shall mean an amount or concentration of a compound according to the present invention which is effective within the context of its administration or use. Thus, the term “effective amount” is used throughout the specification to describe concentrations or amounts of compounds according to the present invention which may be used to produce a favorable change in the disease or condition treated, whether that change is a remission, a decrease in growth or size of cancer or a tumor, a favorable physiological result, a reduction in the growth or elaboration of a microbe, or the like, depending upon the disease or condition treated.

As used herein, the term “preventing effective amount” is used throughout the specification to describe concentrations or amounts of compounds according to the present invention which are prophylactically effective in preventing, reducing the likelihood of infection or delaying the onset of infections in patients caused by microbes.

As used herein, the term “coadministration” or “combination therapy” is used to describe a therapy in which at least two active compounds in effective amounts are used to treat a viral or fungal infection at the same time. Although the term coadministration preferably includes the administration of two active compounds to the patient at the same time, it is not necessary that the compounds be administered to the patient at the same time, although effective amounts of the individual compounds will be present in the patient at the same time.

As used herein, the term “organic solvent” includes but not is limited to 1,2-dichloroethane, dimethoxyethane, diethylene glycol dimethyl ether, tetrahydrofuran, dioxane or diisopropyl ether, hydrocarbons such as hexane, heptane, cyclohexane, toluene or xylene, alcohols such as methanol, ethanol, 1-propanol, 2-propanol, 1-butanol, 2-butanol, tert-butanol or ethylene glycol, ketones such as methyl ethyl ketone or isobutyl methyl ketone, amides such as dimethylformamide, dimethylacetamide or N-methylpyrrolidone, dimethoxyethane, tetrahydrofuran, dioxane, cyclohexane, toluene, xylene, alcohols, e.g. ethanol, 1-propanol, 2-propanol, 1-butanol, tert-butanol and mixtures thereof. 1,2-dichloroethane can be a commonly used organic solvent.

As used herein, the term “treatment”, as used herein, unless otherwise indicated, includes the treatment or prevention of a bacterial infection or protozoa infection as provided in the method of the present invention.

As used herein, unless otherwise indicated, the term “bacterial infection(s)” or “protozoa infections” or “microbial infections” includes bacterial infections and protozoa infections that occur in mammals, fish and birds as well as disorders related to bacterial infections and protozoa infections that may be treated or prevented by administering antibiotics such as the compounds of the present invention. Such bacterial infections, protozoa infections, microbial infections and disorders related to such infections include the following: pneumonia, otitis media, sinusitus, bronchitis, tonsillitis, and mastoiditis related to infection by Streptococcus pneumonlae, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.: urethritis and cervicitis; and sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivits, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis related to infection by Helicobacter pylori or Chlamydia pneumoniae. Bacterial infections and protozoa infections and disorders related to such infections that may be treated or prevented in animals include the following: bovine respiratory disease related to infection by P. haem., P. multocida, Mycoplasma bovis, or Bordetella spp.; cow enteric disease related to infection by E. coli or protozoa (i.e., coccidia, cryptosporidia, etc.); dairy cow mastitis related to infection by Staph. aureus, Strep. uberis, Strep. agalactiae, Strep. dysgalactiae, Kiebsiella spp., Corynebacterium, or Enterococcus spp.; swine respiratory disease related to infection by A. pleuro., P. multocida, or Mycoplasma spp.; swine enteric disease related to infection by E. coli, Lawsonia intracellularis, Salmonella, or Serpulina hyodyisinteriae; cow footrot related to infection by Fusobacterium spp.; cow metritis related to infection by E. coli, cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by Staph. epidermidis, Staph. intermedius, coagulase negative Staph. or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and protozoa infections and disorders related to such infections that may be treated or prevented in accord with the method of the present invention are referred to in J. P. Sanford et al., “The Sanford Guide To Antimicrobial Therapy,” 26th Edition, (Antimicrobial Therapy, Inc., 1996).

### I. Introduction

The increase in multiple antibiotic resistant strains has led to more nosocomial and community-acquired infections. This insurgence of resistant bacterial strains has fueled the search for new antibacterial compounds, including peptides. Antimicrobial peptides are ubiquitous components of prokaryotic and eukaryotic defense mechanisms against invading organisms. Though diverse in amino acid sequence, the amphipathicity and cationic nature of these peptides allow them to interact with and disrupt the bacterial cell wall membrane, which leads to cell death. Effects on intracellular molecules have also been observed, leaving some investigators to conclude that membrane disruption is only a portion of the antimicrobial activity of antimicrobial peptides.

The mode of action to disrupt the membrane with peptide antibiotics is thought to follow the “barrel stave” model or the “carpet” model. Under the barrel stave model, pores are formed across the cell membrane and the cell essentially empties itself into the surrounding environment. Under the carpet model, molecules align and orient themselves parallel to the membrane which causes a disruption of the lipid structure of the membrane and allows the contents of the cell to seep through to the outside. While it seems alluring to attribute the antibacterial effect of these compounds based solely on a physical disruption of the cell membrane, this mechanism alone does not account for the responses that these peptides produce in bacterial cultures.

It has been observed that some cationic peptides have failed to depolarize the bacterial membrane, yet have still effectively inhibited growth. At the minimum inhibitory concentration, these peptides did not disrupt the bacterial membrane. Therefore, some other mechanism of inhibiting the growth of the bacteria must be working. It has also been observed that cationic peptides resulted in changes in gene expression profiles in E. coli in addition to permeabilizing the membrane. Taken together, these observations provide evidence that it is unlikely that the diverse groups of these peptides act by only disruption of the membrane.

It has been discovered that some secondary metabolites from microorganisms may be used as antimicrobials. In the present invention, two secondary metabolites from fluorescent pseudomonads, syringopeptin 25A and rhamnolipids, are examined for their use as antimicrobials. The underlying mode of action of syringopeptin 25A and rhamnolipids is investigated to determine how exposure to the compounds affects the genetic expression profile of Listeria monocytogenes EDGe. As such, gene profiling experiments are described in order to elucidate the mechanism of the antimicrobial activity of syringopeptin 25A and rhamnolipids and to identify new targets for future development of new antimicrobial compounds.

Generally, the present invention provides a composition having compounds, such as syringopeptin 25A and rhamnolipids, with therapeutic properties. Accordingly, the composition having syringopeptin 25A and rhamnolipids can be used as an antimicrobial in order to treat microbial infections. Additionally, the compositions having syringopeptin 25A and rhamnolipids can be used as a prophylactic or therapeutic treatment of cancer, tuberculosis, and others.

In some embodiments, the compounds described herein can be used for the treatment of cancer and/or a microbial infection. Thus, for example, the compounds described herein can be used to treat, prevent the formation of, slow the growth of, or kill cancer cells. In some embodiments, the compounds described herein are administered to a subject suffering from cancer. In one embodiment, the subject is a human. In some embodiments, cancer cells are contacted with one or more of the compounds described herein.

In some embodiments, the compounds described herein can be used to treat a bacterial infection. In some embodiments, the compounds prevent the formation of, slow the growth of, or kill bacteria. In some embodiments, the compounds described herein are administered to a subject suffering from a bacterial infection. In one embodiment, the subject is a human. In some embodiments, bacteria are contacted with one or more compounds described herein. In some embodiments, the bacteria are gram-positive bacteria. In one embodiment, the bacteria is Staphylococcus aureus (methicillin sensitive), Staphylococcus aureus (methicillin resistant), Streptococcus pneumonia (penicillin sensitive), Streptococcus pneumonia (penicillin resistant), Staphylococcus epidermis (multiple drug resistant), Enterococcus faecalis (vancomycin sensitive), or Enterococcus faecium (vancomycin resistant). In some embodiments, the gram-negative bacterium is Haemophilus influenzae.

A. Syringopeptins

Syringopeptins are bacterial secondary metabolites belonging to a class of cyclic lipodepsipeptides produced by certain pathovars of the plant bacterium Pseudomonas syringae. Their peptide portions contain either 22 (SP 22) or 25 (SP 25) amino acids that are predominantly hydrophobic, valine and alanine in particular (see FIG. 1). In FIG. 1, the syringopeptins are shown such that the fatty acids can either 3-hydroxydecanoic or 3-hydroxydodecanoic acid (Abbreviations shown in FIG. 1 for non-standard amino acids are: Dhb is 2,3-dehydroaminobutyric acid, Dab is 2,4-diaminobutyric acid, and aThr is allothreonine). SP22 includes an amino acid sequence of: Dhb-Pro-Val-Val-Ala-Ala-Val-Val-Dhb-Ala-Val-Ala-Ala-Dhb-aThr-Ser-Ala-Dhb-Ala-Dab-Dab-Tyr (SEQ ID NO: 1). SP25 includes an amino acid sequence of: Dhb-Pro-Val-Ala-Ala-Val-Leu-Ala-Ala-Dhb-Val-Dnb-Ala-Val-Ala-Ala-Dhb-aThr-Ser-Ala-Val-Ala-Dab-Dab-Tyr (SEQ ID NO: 2).

Approximately 70% of the chiral residues are of the D configuration, and there are four α,β-unsaturated and two 2,4-diaminobutyric acid residues. An N-terminal residue dehydroaminobutyric acid (Dhb) is N acylated by a 3-hydroxylated fatty acid chain containing either 10 or 12 carbon atoms; these two types of chains are designated A and B homologs and are typically the more abundant and less abundant forms, respectively. The C-terminal carboxyl group is esterified by the hydroxyl group of the allo-Thr residue positioned at the distance of 7 residues, thus forming an eight-membered lactone macrocycle. So far, two SP25 and three SP22 forms have been identified.

Primarily, SPs act as a phytotoxin and function as a virulence factor for P. syringae by inducing necrosis in plant cells. Studies with knock-out mutants have shown that P. syringae deficient in SP production are less virulent, although some diseases may still occur in its absence. SPs have the ability to cause electrolyte leakage by forming pores in plant plasma membranes, thereby promoting transmembrane ion-flux that leads to necrotic symptoms. SPs also display biosurfactant properties, having a critical micelle concentration of 0.9 mM for SP 25A and 0.4 mM for SP 22A. These relatively low critical micelle concentrations may aid in the spread of the phytotoxic organisms on the plant surface by reducing the contact angle of water.

The mode of action of SPs against bacteria is currently unknown; however, it has been observed that SPs form pores in model membranes. One potential mechanism of the antimicrobial activity of SPs is that the molecule first adsorbs onto the cell membrane by partially inserting its hydrophobic acyl chain in between the lipid portions of the phospholipids of the cell membrane. Presumably, the adsorbed monomers then form aggregates that eventually form the pore. After forming aggregates, the hydrophobic portion of the SP molecule unfolds and completely aligns with the lipid tails spanning the membrane and thereby causes the formation of a pore. Once the pore is formed, the cell looses its permeability barrier, ultimately leading to cell death.

In one embodiment, the present invention includes a composition comprising a therapeutically effective amount of a SP as described herein. As such, the composition can include at least one of SP 22 or SP 25. The composition can be configured as any of the formulations for various uses as described herein. Also, the composition can include at least 1% SP, more preferably at least 5% SP, even more preferably at least 10% SP, still more preferably at least 20% SP, and most preferably at least 25% SP. Additionally, the composition can be configured to include less than about 500 ug/mL SP, more preferably less than about 250 ug/mL, even more preferably less than about 100 ug/mL SP, still more preferably less than about 50 ug/mL SP, yet more preferably less than about 25 ug/mL SP, and most preferably less than about 5 ug/mL. However, when combined with a RL, the amount of SP described above can be decreased by half, a quarter, an eighth, or more depending on the amount of RL. Compositions including concentrated SP can be beneficial in uses where the composition is diluted.

In one embodiment, a therapeutic composition can include less than about 1% SP, more preferably less than about 0.5% SP, even more preferably less than about 0.1% SP, still more preferably less than about 0.05% SP, and most preferably less than about 0.025% SP. Additionally, the composition can be configured to include less than about 5 ug/mL SP, more preferably less than about 2.5 ug/mL, even more preferably less than about 1 ug/mL SP, still more preferably less than about 0.5 ug/mL SP, yet more preferably less than about 0.25 ug/mL SP, and most preferably less than about 0.1 ug/mL. Compositions including dilute SP can be beneficial in uses where the composition is not excessively diluted.

Additionally, it is thought that the activity of SPs can also be useful in preventing, inhibiting, or treating other illnesses. As such, the activity of SPs may be useful against cancer, tuberculosis, and other maladies.

B. Rhamnolipids

Rhamnolipids are biosurfactants produced by several strains of Pseudomonads aeruginosa. FIG. 2 is a schematic diagram illustrating a general embodiment of a rhamnolipid (RL), wherein the carbon chain length may be n=4, 6, 8 and R₁ is H or 3-hydroxydecanoate and R₂ is L-rhamnosyl. However, other RLs can be used in the present invention. RLs exhibit antimicrobial activity and are often a mixture of various homologues, depending upon the strain of pseudomonads and the carbon source used during growth. Eleven different RL homologues have been identified in cultures of P. aeruginosa and consist of one or two moieties of rhamnose covalently linked to a 3-0 hydroxy acid, where the chain length of the acid is 8, 10, or 12 carbon atoms (see FIG. 2). In some cases, RLs may also have 3-hydroxy decanoate linked to the fatty acid.

There are several different uses for RLs, but the physiological role of specific RLs is not well understood. RLs help the cell survive by emulsifying hydrocarbons or hydrophobic substrates, making them available for cell metabolism. RLs may be used in bioremediation and biodegradation of both aliphatic and aromatic organic compounds. The addition of RLs to cultures of bacteria increases the biodegradation of hexadecane, octadecane, n-paraffin, phenanthrene, tetradecane, pristine and creosote. The biodegradation is most likely due to the surface-active properties of RLs since they increase the solubility of the hydrocarbons and hence make them readily available for catabolism to the degrading cells. In vivo, RLs bring about structural changes in macrophages so that they cannot associate with the bacteria, thus preventing phagocytosis of the bacteria by the macrophages. In addition, RLs may help the bacterial cells in increasing swarming motility under nutrient limitations. RLs may also play a role as a virulence factor.

RLs are also effective against zoosporic plant pathogens, such as Pythium aphonidermatum, Phytophthara capsici, and Plasmopara lactucae-radicis. RLs render the zoospores non-motile and bring about their lysis in less than a minute at concentrations of 5-30 μg/mL.

Another potential use of RLs may be to use their antimicrobial activity to cripple other microbes that compete for the same pool of nutrients. RLs may increase the surface hydrophobicity of the cells by removing lipopolysaccharides from the cell wall and therefore improving the association of more hydrophobic substrates which help to destroy the integrity of the cell membrane.

In one embodiment, the present invention includes a composition comprising a therapeutically effective amount of a RL as described herein. The composition can be configured as any of the formulations for various uses as described herein. Also, the composition can include at least 1% RL, more preferably at least 5% RL, even more preferably at least 10% RL, still more preferably at least 20% RL, and most preferably at least 25% RL. Additionally, the composition can be configured to include less than about 500 ug/mL RL, more preferably less than about 250 ug/mL RL, even more preferably less than about 100 ug/mL RL, still more preferably less than about 50 ug/mL RL, yet more preferably less than about 25 ug/mL RL, and most preferably less than about 5 ug/mL RL. However, when combined with a SP, the amount of RL described above can be decreased by half, a quarter, an eighth, or more depending on the amount of SP. Compositions including concentrated RL can be beneficial in uses where the composition is diluted.

In one embodiment, a therapeutic composition can include less than about 1% RL, more preferably less than about 0.5% RL, even more preferably less than about 0.1% RL, still more preferably less than about 0.05% RL, and most preferably less than about 0.025% RL. Additionally, the composition can be configured to include less than about 5 ug/mL RL, more preferably less than about 2.5 ug/mL RL, even more preferably less than about 1 ug/mL RL, still more preferably less than about 0.5 ug/mL RL, yet more preferably less than about 0.25 ug/mL RL, and most preferably less than about 0.1 ug/mL RL. Compositions including dilute RL can be beneficial in uses where the composition is not excessively diluted.

Additionally, it is thought that the activity of RLs can also be useful in preventing, inhibiting, or treating other illnesses. As such, the activity of RLs may be useful against cancer, tuberculosis, and other maladies.

C. Membrane Permeability and Antimicrobial Activity

Prior to the present invention, the antimicrobial activity of RLs and SP 25A were both generally attributed to their disruption of the microbial plasma membrane. In the present invention, the relationship between membrane permeabilization and the antimicrobial activity of SP 25A and RLs was investigated by examining the correlation between compromised cell membranes and an inhibition of microbial growth.

In the present invention, membrane permeabilization of microbes was measured while being exposed to MICs of SP 25A or RLs (see Example 3). Membrane permeabilization was determined through following the change in fluorescence of a dye, propidium iodide (“PI”). PI is unable to pass through the cell membrane unless the membrane has been physically compromised. Once inside the cell, the intercalation of the PI with the DNA of the cell causes the fluorescent properties of the dye to change. This change in fluorescence can be measured. Therefore, an uptake of PI indicates an increase in membrane permeabilization.

As discussed in more detail below, it was determined that the permeability of the microbial plasma membrane does not necessarily correlate with inhibiting growth of the microbial.

D. Gene Profiling

Gene profiling is one method that can be utilized to elucidate the mechanism of an antimicrobial compound. Gene profiling measures the change in the cell's regulation of expressed genes. In the present invention, gene profiling was used to determine the effect that exposure to SP 25A and RLs had upon the regulation of genes in L. monocytogenes. Several biochemical pathways essential to survival of the bacteria were either up regulated or down regulated in response to the exposure of the bacteria to sub-MIC doses of SP 25A and RLs. This information was used to help elucidate the mechanism by which SP 25A and RLs possess antimicrobial activity. Several new antimicrobial targets were identified (see Example 5).

E. Lack of Toxicity of RLs and SP 25A in Mammalian Cells

It is important for an effective therapeutic to lack toxicity in the patient to whom it is being administered. In the present invention, the toxicity of RLs and SP 25A was determined in three mammalian cell lines. Up to average MICs, neither RLs or SP 25A exhibited any toxicity towards the mammalian cells. The three mammalian cells tested represented important and varied mammalian tissue types, lung, gut and kidney. Therefore, under circumstances where there is a systemic microbial infection or a cancer that has spread throughout the body of a patient, RLs, SP 25A, and a mixture of RLs and SP 25A possess substantial therapeutic potential (see Example 4).

### II. Antimicrobial Properties of RLs and SPs

RLs and SP 25A were screened to test their antimicrobial potential. They were tested against 27 different organisms, including gram-positive bacteria, gram-negative bacteria, molds, multiple drug resistant human pathogens, food spoilage organisms, bacterial spores, and fermentative bacteria (see Table 1, which includes a list of bacteria used for antimicrobial screening and their growth conditions.). The IR and MIC for SP 25A and RLs was determined for each microbe (see Table 2, which includes MICs and mean IR's of SP 25A and rhamnolipids against screened organisms). Both SP 25A and RLs inhibited the growth of all the gram-positive organisms that were tested. Mycobacterium smegmatis, a surrogate test organism for Mycobacterium tuberculosis, was also inhibited. SP 25A inhibited the growth of multiple antibiotic resistant strains of S. aureus and Enterococcus faecalis. In the present invention, the inhibition of spore germination of bacterial spores from Bacillus subtilis and Clostridium sporogenes was observed upon exposure to SP 25A and RLs (see Table 2).

FIG. 4 shows the inhibition rate for L. monocytogenes with RLs alone (0, 0.5, 1, 1.5, 3 and 6 μg/mL), and with rhamnolipids combined with SP 25A (3 μg/mL). SP 25A and RLs were combined and tested together against L. monocytogenes in order to determine what the effect on the overall rate of inhibition would be for a mixture of the compounds versus RLs used alone. A synergistic increase on the rate of inhibition was observed while testing a mixture of the two compounds on L. monocytogenes (see FIG. 4). The inhibition rate for SP 25A was approximately one-sixth the inhibition rate for RLs when each compound was tested by itself on L. monocytogenes (see Table 2). However, when used in concert, a synergistic effect was observed on the inhibition rate against L. monocytogenes (see FIG. 4). As such, the individual or combined amounts of SP and RL included in a composition of the present invention can be substantially similar to those shown to be effective in FIG. 4.

Accordingly, FIG. 4 depicts the differences in inhibition rates for varying concentrations of RL against the same concentrations of RL with the addition of a constant amount of SP 25A. The rate of inhibition for the mixture of SP25A and a given concentration of RLs is always greater than the same concentration of RLs used alone. This difference has broad utility in the treatment of microbial infections, treatment of a neoplastia condition or herbicidal applications. Cells that are more resistant to treatment by one or both of the compounds individually will likely be more susceptible to treatment from a mixture of SP 25A and RLs. This synergistic interaction between RLs and SP 25A allows for the use of lower concentrations of SP 25A and RLs in potential pharmaceutical compositions used for treatment of a microbial infection, a neoplastia, or an herbicidal application. The discovery of the synergistic effect also may allow for treatment of organisms previously resistant to treatment by using the individual compounds.

In order to determine the antimicrobial efficacy of RLs and SPs they were tested against several bacterial strains, some with multiple drug resistance. The multiple drug resistant bacterial strains used were E. faecalis and S. aureus. They both possessed resistance to gentamicin, vancomycin and teicoplanin. Additionally, the S. aureus used was only intermediately susceptible to vancomycin, as well as possessing resistance to methicillin. A combination of RLs and SPs were tested against L. monocytogenes.

Both SP 25A and RLs compromised the membrane of all the gram-positive bacteria with RLs acting significantly faster (3-433 times depending upon the organism tested) than SP 25A (see Table 2). Inhibition was also confirmed by the microbroth dilution method to determine the minimum inhibitory concentration (see Example 2). Both compounds inhibited all the gram-positive organisms tested, as well as Flavobacterium devorans with MICs ranging from 3 μg/mL to 32 μg/mL (see Table 2). Both compounds inhibited Mycobacterium smegmatis, Bacillus subtilis spores, and Clostridium sporogenes spores with an MIC of 4 μg/mL. The inhibition of spore germination from C. sporogenes and B. subtilis had, henceforth, not been observed.

RLs had a higher inhibitory rate than SP 25A, yet RLs were unable to inhibit growth even at a concentration of 60 μg/mL (see Examples 2 and 3). The lack of a positive correlation between the ability of the RLs to permeabilize the cellular membrane and their antimicrobial activity may be explained under several theories. One explanation of the lack of positive correlation is that the biochemical changes brought about by RLs were overcome by a stress response from the targeted cells that repaired the compromised membrane. However the cells could not repair the changes brought about by SP 25A. This reveals that either the RLs and SP 25A have different modes of action on the cell membrane or possibly that SP 25A has multiple modes of action (i.e., it may act on multiple cellular targets).

FIGS. 6A includes a graph that illustrates the amount of membrane permeabilization in response to SP 25A and RLs. Membrane permeabilization was determined by the increase in fluorescence (RFU) with PI uptake during treatment of L. monocytogenes with SP 25A and RLs over a period of 120 min. When L. monocytogenes was exposed to sub-MIC doses of RLs and SP 25A, RLs induced PI uptake while SP 25A did not (see FIG. 6A).

FIG. 6B includes a graph that illustrates the amount of cell growth in response to being treated with SP 25A, RLs and saline (control) over a period of 120 min. It was shown that SP 25A completely inhibited cell growth, and RLs partially inhibited cell growth in comparison to the saline control. The lack of positive correlation between membrane permeabilization (FIG. 6A) and inhibition of cell growth (FIG. 6B) of SP 25A inferred that the pore forming model alone was not be responsible for all of the antimicrobial properties of SP 25A.

Through gene profiling experiments, the cellular response to treatment with SP 25A and RLs was determined (see Example 5). The cell permeabilization, cell density, and the transcription profile caused by the two compounds were strikingly different. RLs caused an increase of ˜53% in membrane permeabilization, and inhibited growth by ˜47% (see FIGS. 6A and 6B). Conversely, SP 25A permeabilized the membrane by ˜2%, but led to complete inhibition of cell growth, demonstrating the lack of a positive correlation between membrane permeabilization and cell growth inhibition (see FIGS. 6A and 6B). These observations for the effect of SP 25A confirmed that additional mechanisms beyond membrane disruption are involved for bacterial inhibition. In contrast to the activity of SP 25A, the membrane disruption caused by RLs was directly linked to the observed bacterial inhibition. RLs disrupted the plasma membrane enough for the tested microbes to take up PI, yet the microbes still retained the ability to replicate and growth was not completely arrested.

The foregoing experiments demonstrate that SP 25A and RLs compromise the membrane of gram-positive bacteria with MICs of ≦8 μg/mL. Additionally, SP 25A and RLs act synergistically to inhibit L. monocytogenes resulting in lower MICs for each compound when used in combination. Considering the inhibition of multiple drug resistant strains of enterococci and staphylococci, Mycobacterium, Bacillus spores, and the lack of toxicity towards mammalian cells, SP 25A and SP 25A in combination with RLs are a very promising antimicrobial therapeutic.

The following preparations and examples are given to enable those skilled in the art to more clearly understand and to practice the present invention. They should not be considered as limiting the scope of the invention, but merely as being illustrative and representative thereof.

### III. Anti-Tuberculosis Activity

Tuberculosis (TB) is an infectious disease that usually attacks the lungs, but is capable of attacking most other parts of the body. Tuberculosis is spread from person to person through the air. When individuals infected with TB cough, laugh, sneeze, or talk, TB bacteria can be spread into the air. If a second person inhales TB bacteria, a possibility exists that the second person also will become infected with tuberculosis. However, repeated contact typically is required for infection.

Medical experts estimate that about 10 million Americans are infected with TB bacteria, and about 10 percent of these people will develop active TB in their lifetime. However, TB is an increasing worldwide problem, especially in Africa. It is estimated that, worldwide, about one billion people will become newly infected, over 150 million people will contract active TB, and 36 million people will die between now and 2020 unless TB control is improved.

An individual infected with TB, but not suffering from TB disease, i.e., has latent TB, can be administered a preventive therapy. Preventive therapy kills bacteria in order to prevent a case of active TB. The usual treatment for latent TB is a daily dose of isoniazid (“INH”).

If an individual has TB disease, i.e., has active TB, the individual typically is administered a combination of several drugs. It is very important, however, that the individual continue a correct treatment regimen for the full length of the treatment. If the drugs are taken incorrectly, or stopped, the individual can suffer a relapse and will be able to infect others with TB.

When an individual becomes sick with TB a second time, the TB infection may be more difficult to treat because the TB bacteria have become drug resistant, i.e., TB bacteria in the body are unaffected by some of the drugs that are commonly used to treat TB. Multidrug-resistant tuberculosis (“MDR TB”) is a very dangerous form of tuberculosis. In particular, some TB bacteria become resistant to the effects of various anti-TB drugs, and these resistant TB bacteria then can cause TB disease. Like regular TB, MDR TB can be spread to others.

Compounds according to the present invention may be used in pharmaceutical compositions having biological/pharmacological activity for the treatment of, for example, TB, including latent TB, active TB, and MDR TB, as well as a number of other conditions and/or disease states, as intermediates in the synthesis of compounds exhibiting biological activity as well as standards for determining the biological activity of the present compounds as well as other biologically active compounds. In some applications, the present compounds may be used for treating microbial infections, especially including infections of the Mycobacterium infections. These compositions comprise an effective amount of any one or more of the compounds disclosed herein above as well as in the examples herein below, optionally in combination with a pharmaceutically acceptable additive, carrier or excipient.

A further aspect of the present invention relates to the treatment of TB, including latent TB, active TB and MDR TB, comprising administering to a patient in need thereof an effective amount of a compound as described herein above and herein below, optionally in combination with a pharmaceutically acceptable additive, carrier or excipient. The present invention also relates to methods for inhibiting the growth of Mycobacterium in general and TB specifically, including latent TB, active TB and MDR TB or other Mycobacterium, comprising exposing the TB to an inhibitory or therapeutically effective amount or concentration of at least one of the disclosed compounds or a mixture of the disclosed compounds. This method may be used therapeutically, in the treatment of TB, including latent TB, active TB, and MDR TB or in comparison tests such as assays for determining the activities of related analogs as well as for determining the susceptibility of a patient's TB to one or more of the compounds according to the present invention. Primary utility resides in the treatment of TB, including latent TB, active TB and MDR TB, including TB in immuno-compromised individuals, among others.

A therapeutic aspect according to the present invention relates to methods for treating TB, including latent TB, active TB, and MDR TB and TB infections in animal or human patients, and in embodiments, comprising administering therapeutically effective amounts or concentrations of one or more of the compounds according to the present invention to inhibit the growth or spread of or to actually shrink the TB infection, cause the TB infection to revert to a latent state, or effectively overcome a TB infection in the animal or human patient being treated.

In the present invention, M. smegmatis was used as a surrogate organism in place of TB bacteria. M. smegmatis and TB are from the same genus and share many of the same essential physiological attributes and possess most of the same biochemical pathways. The compounds of the present invention were shown to inhibit the growth of M. smegmatis (see Table 2). The MIC against M. smegmatis was determined for SP 25A and for RLs. Individually, each compound was able to inhibit the growth of M. smegmatis, each having a MIC of 4 μg/mL (see Table 2).

Given the synergistic effect upon the inhibition rate of the compounds upon the growth of L. monocytogenes (see FIG. 4) and the general ability of the compounds of the present invention to inhibit microbes that do not possess the additional permeable barrier of the gram-negative bacteria, it is highly likely that a mixture of the compounds of the present invention would individually be even more effective as a therapeutic against M. tuberculosis infection. Therefore, it is likely that the compounds of the present invention may be used to fight inactive, active, or MDR TB.

A. In Vitro Activity and Selectivity

Compounds of the present invention may be tested for MIC against M. tuberculosis H_{37Rv} in an axenic medium and for cytotoxicity against African green monkey kidney cell line (“VERO cells”). Compounds are routinely tested for cytotoxicity in the ITR using VERO cells (C. L. Cantrell et al., J. Nat. Prod., 59:1131-36 (1996); G. C. Mangalindan et al., Planta Med., 66:364-5 (2000)). Compounds of the present invention can be tested against VERO cells at concentrations less than or equal to 1% of the maximum achievable stock concentration. This may result in a final DMSO concentration of less than or equal to 1% v/v, which is often the maximum non-cytotoxic concentration.

Testing at very high concentrations allows for the recognition of high degrees of selectivity. Repeat testing can be performed for compounds for which the IC₅₀ is less than or equal to the lowest tested concentration, when this concentration also is above the MIC for M. tuberculosis. After 72 hours exposure, viability may be assessed on the basis of cellular conversion of MTS into a soluble formazan product using the Promega CellTiter 96 aqueous non-radioactive cell proliferation assay. Rifampin, clarithromycin, cethromycin, and telithromycin can be included as controls.

For compounds of the present invention having an IC₅₀:MIC ratio greater than >10, cytotoxicity can be repeated using the J774.1 macrophage cell line because these are used in the macrophage assay and typically are all more sensitive than VERO cells.

Compounds of the present invention for which the IC₅₀:MIC(SI) ratio is greater than 10 can be tested for activity against M. tuberculosis Erdman (ATCC 35801) in monolayers of J774.1 murine macrophages (EC₉₉ and EC₉₉; lowest concentration effecting a 90% and 99% reduction in colony forming units at 7 days compared to drug-free controls) at 4-fold or 5-fold concentrations with the lowest concentration just below the MIC.

Compounds of the present invention may also be evaluated for MIC vs. M. tuberculosis H_{37Rv} using the microplate Alamar Blue assay (MABA) described in (L. Collins et al., Antimicrob. Agents Chemother., 41:1004-9 (1997)) except that 7H12 media, rather than 7H9+glycerol+casitone+OADC, is used. The use of this and other redox reagents, such as MTT, have shown excellent correlation with cfu-based and radiometric analyses of mycobacterial growth. The MIC is defined as the lowest concentration effecting a reduction in florescence (or luminescence) of 90% relative to controls. Isoniazid and rifampin can be included as positive quality control compounds for each test, with expected MIC ranges of 0.025-0.1 and 0.06-0.125 μg/ml, respectively. MBCs are determined by subculture onto 7H11 agar just prior to addition of Alamar Blue and Tween 80 reagents to the test wells. The MBC is defined as the lowest concentration reducing cfu by 99% relative to the zero time inoculum.

Anti-tuberculosis therapeutic compositions of the present invention may include the combination of RLs and SP 25A in a pharmaceutically acceptable composition (see Examples 11-15). Therapeutic compositions of the present invention may be administered to subjects infected with latent, active, or MDR tuberculosis (see Examples 16-18).

B. Agar Proportion Method and BACTEC

Additionally, the anti-tuberculosis therapeutic compositions of the present invention may include the combination of RLs and SP 25A in a pharmaceutically acceptable composition can be tested using other methods to determine efficacy for treating and/or preventing tuberculosis. Such testing can be performed by the Agar Proportion Method as described by the National Center for Clinical Laboratory Services of India. The Agar Proportion Method is relatively inexpensive and simple, and can provide results in 3 weeks. Also, the BACTEC system (Becton Dickinson) and E-test method can also be used to test the RLs and SP 25A compositions against tuberculosis. Agar Proportion Method and BACTEC system, and E-test method for testing the anti-tuberculosis of RLs and SP 25A is well within the ability of one or ordinary skill in the art. Additional information regarding the Agar Proportion Method, BACTEC system, and E-test method can be found in the following references: Wagner A and Mills K, Testing of Mycobacterium Tuberculosis Susceptibility to Ethambutol. Isoniazid, Rifampicin and Streptomycin by using E-test, J Clin Microbiol 34; 1672 (1996); Hazbon M H, Orozco M S, Labrada L A, Tovar R, Weigle K A, Wagner A, Evaluation of E-test for Susceptibility Testing of Multi-Drug Resistant Isolates of Mycobacterium Tuberculosis, J Clin Microbiol 38:4599 (2000); Varma M, Kumar S, Kumar A, and Bose M, Comparison of Etest and Agar Proportion Method of Testing Drug Susceptibility of M. Tuberculosis, Ind J Tub 490:217 (2002); Joloba M L, Majaksouzian S, Jacobs M R, Evaluation of Etest for Susceptibility of Mycobacterium Tuberculosis, Int J Tuber Lung Dis 2:751 (1998), which are incorporated herein by specific reference. Moreover, additional information regarding the Agar Proportion Method, BACTEC system, and E-test is provided in the following examples.

### IV. Antineoplastic Activity

Cancer is a leading cause of death in the United States. Despite significant efforts to find new approaches for treating cancer, the primary treatment options remain surgery, chemotherapy and radiation therapy, either alone or in combination. Surgery and radiation therapy, however, are generally useful only for fairly defined types of cancer, and are of limited use for treating patients with disseminated disease. Chemotherapy is the method that is generally used in treating patients with metastatic cancer or diffuse cancers such as leukemias. Although chemotherapy can provide a therapeutic benefit, it often fails to result in cure of the disease due to the patient's cancer cells becoming resistant to the chemotherapeutic agent. Due, in part, to the likelihood of cancer cells becoming resistant to a chemotherapeutic agent, such agents are commonly used in combination with other compounds to treat patients.

Compounds according to the present invention may be used in pharmaceutical compositions having biological/pharmacological activity for the treatment of, for example, neoplasia, including cancer, as well as a number of other conditions and/or disease states, as intermediates in the synthesis of compounds exhibiting biological activity as well as standards for determining the biological activity of the present compounds as well as other biologically active compounds. In some applications, the present compounds may be used for treating microbial infections, especially including viral infections. These compositions comprise an effective amount of any one or more of the compounds disclosed hereinabove, optionally in combination with a pharmaceutically acceptable additive, carrier or excipient.

A further aspect of the present invention relates to the treatment of neoplasia, including cancer, comprising administering to a patient in need thereof an effective amount of a compound as described hereinabove, optionally in combination with a pharmaceutically acceptable additive, carrier or excipient. The present invention also relates to methods for inhibiting the growth of neoplasia, including a malignant tumor or cancer comprising exposing the neoplasia to an inhibitory or therapeutically effective amount or concentration of at least one of the disclosed compounds. This method may be used therapeutically, in the treatment of neoplasia, including cancer or in comparison tests such as assays for determining the activities of related analogs as well as for determining the susceptibility of a patient's cancer to one or more of the compounds according to the present invention. Primary utility resides in the treatment of neoplasia, including cancer, especially including lung cancer, breast cancer and prostate cancer, among others.

A therapeutic aspect according to the present invention relates to methods for treating neoplasia, including benign and malignant tumors and cancer in animal or human patients, and in embodiments, cancers which have developed drug resistance, including, for example, multiple drug resistant breast cancer comprising administering therapeutically effective amounts or concentrations of one or more of the compounds according to the present invention to inhibit the growth or spread of or to actually shrink the neoplasm in the animal or human patient being treated.

Cancers which may be treated using compositions according to the present invention include, for example, stomach, colon, rectal, liver, pancreatic, lung, breast, cervix uteri, corpus uteri, ovary, prostate, testis, bladder, renal, brain/central nervous system, head and neck, throat, Hodgkins disease, non-Hodgkins leukemia, multiple myeloma leukemias, skin melanoma, acute lymphocytic leukemia, acute mylogenous leukemia, Ewings Sarcoma, small cell lung cancer, choriocarcinoma, rhabdomyosarcoma, Wilms Tumor, neuroblastoma, hairy cell leukemia, mouth/pharynx, oesophagus, larynx, melanoma, kidney and lymphoma, among others.

In the present methods, in certain embodiments, it has been found advantageous to co-administer at least one additional anti-neoplasia agent for the treatment of neoplasia, including cancer. In these aspects according to the present invention, an effective amount of one or more of the compounds according to the present invention is co-administered along with an effective amount of at least one additional anti-neoplasia/anticancer agent such as, for example cyclophosphamide.

A. Treatment of Carcinomas and Tumors

Carcinomas that can be treated using the compounds, compositions and methods described herein include colorectal carcinoma, gastric carcinoma, signet ring type, esophageal carcinoma, intestinal type, mucinous type, pancreatic carcinoma, lung carcinoma, breast carcinoma, renal carcinoma, bladder carcinoma, prostate carcinoma, testicular carcinoma, ovarian carcinoma, endometrial carcinoma, thyroid carcinoma, liver carcinoma, larynx carcinoma, mesothelioma, neuroendocrine carcinomas, neuroectodermal tumors, melanoma, gliomas, neuroblastomas, sarcomas, leiomyosarcoma, MFII, fibrosarcoma, liposarcoma, MPNT, chondrosarcoma, and lymphomas.

To treat cancer the compounds of the present invention are administered intravenously, enterally (e.g., as an enteric coated tablet form), by aerosol, orally, transdermally, transmucosally, intrapleurally, intrathecally, or by other suitable routes.

The following preparations and examples are given to enable those skilled in the art to more clearly understand and to practice the present invention. They should not be considered as limiting the scope of the invention, but merely as being illustrative and representative thereof.

### V. Compositions

The compounds of the present invention can be formulated into a pharmaceutically acceptable formulation. Such a composition can be useful to prevent, alleviate or eliminate the symptoms and/or organisms associated with microbial infections, and thereby can be used as a prophylactic or treatment for microbial infections. Additionally, such a composition can be useful as to prevent, alleviate or eliminate the symptoms and/or organisms associated with cancer, tuberculosis, and other maladies, and thereby can be used as a prophylactic or treatment for such maladies.

In embodiments of the present invention, the pharmaceutical composition comprises an active component and inactive components. The active components are syringopeptins (e.g., SP 25A) and/or rhamnolipids. The inactive components are selected from the group consisting of excipients, carriers, solvents, diluents, stabilizers, enhancers, additives, adhesives, and combinations thereof.

The amount of the compound in a formulation can vary within the full range employed by those skilled in the art. Typically, the formulation will contain, on a weight percent basis, from about 0.01-99.99 weight percent of the compounds of the present invention based on the total formulation, with the balance being one or more suitable pharmaceutical excipients. Preferably, the compounds are present at a level of about 1-80 weight percent.

A pharmaceutical composition of the present invention may optionally contain, in addition to a pharmacological agent, at least one other therapeutic agent useful in the treatment of a condition. Such other compounds may be of any class of drug or pharmaceutical agent, including but not limited to antibiotics, anti-parasitic agents, antifungal agents, anti-viral agents, and anti-tumor agents. When administered with anti-parasitic, anti-bacterial, anti-fungal, anti-tumor, anti-viral agents, and the like, pharmacological agents may be administered by any method and route of administration suitable to the treatment of the condition, typically as pharmaceutical compositions.

Preparations include sterile aqueous or non-aqueous solutions, suspensions and emulsions. Examples of non-aqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents and inert gases and the like. Those of skill in the art can readily determine the various parameters for preparing these pharmaceutical compositions without resort to undue experimentation.

Pharmacological compositions may be prepared from water-insoluble compounds, or salts thereof, such as aqueous base emulsions. In such embodiments, the pharmacological composition will typically contain a sufficient amount of pharmaceutically acceptable emulsifying agent to emulsify the desired amount of the pharmacological agent. Useful emulsifying agents include, but are not limited to, phosphatidyl cholines, lecithin, and the like.

Additionally, the compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Furthermore, pharmacological agent compositions may, though not always, contain microbial preservatives. Microbial preservatives that may be employed include, but are not limited to, methylparaben, propylparaben, and benzyl alcohol. The microbial preservative may be employed when the pharmacological agent formulation is placed in a vial designed for multi-dose use. Pharmacological agent compositions for use in practicing the subject methods may be lyophilized using techniques well known in the art.

The compositions may also include components, such as cyclodextrins, to enhance the solubility of one or more other components included in the compositions. Cyclodextrins are widely known in the literature to increase the solubility of poorly water-soluble pharmaceuticals or drugs and/or enhance pharmaceutical/drug stability and/or reduce unwanted side effects of pharmaceuticals/drugs. For example, steroids, which are hydrophobic, often exhibit an increase in water solubility of one order of magnitude or more in the presence of cyclodextrins. Any suitable cyclodextrin component may be employed in accordance with the present invention. The useful cyclodextrin components include, but are not limited to, those materials which are effective in increasing the apparent solubility, preferably water solubility, of poorly soluble active components and/or enhance the stability of the active components and/or reduce unwanted side effects of the active components. Examples of useful cyclodextrin components include, but are not limited to: β-cyclodextrin, derivatives of β-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-β-cyclodextrin, sulfobutylether-β-cyclodextrin, and the like and mixtures thereof.

The specific cyclodextrin component selected should have properties acceptable for the desired application. The cyclodextrin component should have or exhibit reduced toxicity, particularly if the composition is to be exposed to sensitive body tissue, for example, eye tissue, etc. Very useful β-cyclodextrin components include β-cyclodextrin, derivatives of β-cyclodextrin and mixtures thereof. Particularly useful cyclodextrin components include sulfobutylether β-cyclodextrin, hydroxypropyl cyclodextrin and mixtures thereof. Sulfobutylether β-cyclodextrin is especially useful, for example, because of its substantially reduced toxicity.

The pharmaceutically acceptable excipients, such as vehicles, adjuvants, carriers or diluents, are readily available to the public. Examples of suitable excipients can include, but are not limited to, the following: acidulents, such as lactic acid, hydrochloric acid, and tartaric acid; solubilizing components, such as non-ionic, cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and zein; buffering components, such as sodium phosphate, malic acid, and potassium citrate; chelating components, such as EDTA, malic acid, and maltol; coating components, such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as colloidal silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitostearate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing components, such as cyclodextrins, albumin, xanthan gum; and tonicity components, such as glycerol, dextrose, potassium chloride, and sodium chloride; and mixture thereof. Excipients include those that alter the rate of absorption, bioavailability, or other pharmacokinetic properties of pharmaceuticals, dietary supplements, alternative medicines, or nutraceuticals.

Other examples of suitable excipients, binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

In some embodiments, the compounds in the compositions may be present as a pharmaceutically acceptable salt. The term “pharmaceutically acceptable salts” includes salts of the composition, prepared, for example, with acids or bases, depending on the particular substituents found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galactunoric, salicylic, formic, naphthalene-2-sulfonic, and the like. Still other suitable acids include amino acids such as arginate, aspartate, glutamate, and the like.

In general, pharmaceutically acceptable carriers for are well-known to those of ordinary skill in the art. This carrier can be a solid or liquid and the type is generally chosen based on the type of administration being used. Suitable pharmaceutical carriers are, in particular, fillers, such as sugars, for example lactose, sucrose, mannitol or sorbitol, cellulose preparations and/or calcium phosphates, for example tricalcium phosphate or calcium hydrogen phosphate, furthermore, binders such as starch paste, using, for example, corn, wheat, rice or potato starch, gelatin, tragacanth, methylcellulose and/or polyvinylpyrrolidone, if desired, disintegrants, such as the abovementioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example, to identify or to indicate different doses of active ingredient, may be added to the tablets or sugar-coated tablet coatings.

Additional pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Additional formulations for use in the present invention can be found in Remington's Pharmaceutical Sciences (Mack Publishing Company, Philadelphia, Pa., 17th ed. (1985)), which is incorporated herein by reference. Moreover, for a brief review of methods for drug delivery, see, Langer, Science 249:1527-1533 (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. Other examples of suitable pharmaceuticals are listed in 2000 Med Ad News 19:56-60 and The Physicians Desk Reference, 53rd edition, 792-796, Medical Economics Company (1999), both of which are incorporated herein by reference.

In general, compounds of this invention can be administered as pharmaceutical compositions by any one of the following routes: oral, systemic (e.g., transdermal, intranasal or by suppository), or parenteral (e.g., intramuscular, intravenous or subcutaneous) administration. One manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions. Another manner for administering compounds of this invention is inhalation. This is an effective method for delivering a therapeutic agent directly to the respiratory tract for the treatment of diseases such as asthma and similar or related respiratory tract disorders (see U.S. Pat. No. 5,607,915, herein incorporated by reference).

Pharmaceutical compositions according to the present invention for enteral or parenteral administration are, for example, those in unit dose forms, such as sugar-coated tablets, tablets, capsules, gel caps, caplets, or suppositories, and ampoules. The compositions may also be in sublingual dosages, sustained release formulations and elixirs. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

Suitable rectally utilizable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 (herein incorporated by reference) describes a pharmaceutical formulation having particles in the size range from 10 to 1,000 nm in which the active material is supported on a crosslinked matrix of macromolecules. U.S. Pat. No. 5,145,684 (herein incorporated by reference) describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The choice of formulation depends on various factors such as the mode of drug administration and bioavailability of the drug substance. For delivery by inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist which is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

According to the methods of the present invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced using any mode of administration that is medically acceptable, meaning any mode that produces effective levels of the active composition without causing clinically unacceptable adverse effects.

The administration may be localized (i.e., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be used for some treatments because of the convenience to the patient as well as the dosing schedule. Compositions suitable for oral administration may be presented as discrete units such as capsules, pills, cachettes, tables, or lozenges, each containing a predetermined amount of the active compound. Other oral compositions include suspensions in aqueous or non-aqueous liquids such as a syrup, an elixir, or an emulsion.

For injection, the compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds can be formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulator agents such as suspending, stabilizing and/or dispersing agents.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds can be encapsulated in a vehicle such as liposomes that facilitates transfer of the bioactive molecules into the targeted tissue, as described, for example, in U.S. Pat. No. 5,879,713 to Roth et al. and Woodle, et al., U.S. Pat. No. 5,013,556, the contents of which are hereby incorporated by reference. The compounds can be targeted by selecting an encapsulating medium of an appropriate size such that the medium delivers the molecules to a particular target. For example, encapsulating the compounds within microparticles, preferably biocompatible and/or biodegradable microparticles, which are appropriate sized to infiltrate, but remain trapped within, the capillary beds and alveoli of the lungs can be used for targeted delivery to these regions of the body following administration to a patient by infusion or injection.

Microparticles can be fabricated from different polymers using a variety of different methods known to those skilled in the art. The solvent evaporation technique is described, for example, in E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984). The hot-melt microencapsulation technique is described by E. Mathiowitz, et al., Reactive Polymers, 6, 275 (1987). The spray drying technique is also well known to those of skill in the art. Spray drying involves dissolving a suitable polymer in an appropriate solvent. A known amount of the compound is suspended (insoluble drugs) or co-dissolved (soluble drugs) in the polymer solution. The solution or the dispersion is then spray-dried. Microparticles ranging between 1-10 microns are obtained with a morphology which depends on the type of polymer used.

Microparticles made of gel-type polymers, such as alginate, can be produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath to allow sufficient time for gelation to occur. Microparticle particle size is controlled by using various size extruders or varying either the nitrogen gas or polymer solution flow rates.

Particle size can be selected according to the method of delivery which is to be used, typically size IV injection, and where appropriate, entrapment at the site where release is desired.

In one embodiment, the liposome or microparticle has a diameter which is selected to lodge in particular regions of the body. For example, a microparticle selected to lodge in a capillary will typically have a diameter of between 10 and 100, more preferably between 10 and 25, and most preferably, between 15 and 20 microns. Numerous methods are known for preparing liposomes and microparticles of any particular size range. Synthetic methods for forming gel microparticles, or for forming microparticles from molten materials, are known, and include polymerization in emulsion, in sprayed drops, and in separated phases. For solid materials or preformed gels, known methods include wet or dry milling or grinding, pulverization, classification by air jet or sieve, and the like.

Embodiments may also include administration of at least one pharmacological agent using a pharmacological delivery device such as, but not limited to, pumps (implantable or external devices), epidural injectors, syringes or other injection apparatus, catheter and/or reservoir operatively associated with a catheter, injection etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert at least one pharmacological agent into the delivery device for administration of the pharmacological agent to a subject and include, but are not limited to, vials, ampules, tubes, capsules, bottles, syringes and bags.

Sterile injectable forms of the compositions of this invention may be aqueous or a substantially aliphatic suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Pharmacological agents may be delivered transdermally, by a topical route, formulated as applicator sticks, solutions, suspensions, emulsions, gels, creams, ointments, pastes, jellies, paints, powders, and aerosols. For example, embodiments may include a pharmacological agent formulation in the form of a discrete patch or film or plaster or the like adapted to remain in intimate contact with the epidermis of the recipient for a period of time. For example, such transdermal patches may include a base or matrix layer, e.g., polymeric layer, in which one or more pharmacological agent(s) are retained. The base or matrix layer may be operatively associated with a support or backing. Pharmacological agent formulations suitable for transdermal administration may also be delivered by iontophoresis and may take the form of an optionally buffered aqueous solution of the pharmacological agent compound. Suitable formulations may include citrate or bis/tris buffer (pH 6) or ethanol/water and contain a suitable amount of active ingredient. Topical application for the lower intestinal tract can be effected in a rectal suppository formulation (see above) or in a suitable enema formulation. Topically-transdermal patches may also be used.

For other topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Depending on the mode of administration, the pharmaceutical composition will preferably comprise from 0.05 to 99% by weight, more preferably from 0.1 to 70% by weight of the active ingredient, and, from 1 to 99.95% by weight, more preferably from 30 to 99.9 weight % of a pharmaceutically acceptable carrier, all percentages being based on the total composition.

### VI. Therapeutic Dosages

The compositions of the present invention may be given in dosages, generally at the maximum amount while avoiding or minimizing any potentially detrimental side effects. The compositions can be administered in effective amounts, alone or in a cocktail with other compounds, for example, other compounds that can be used to treat cancer or microbial infections. An effective amount is generally an amount sufficient to inhibit an associated cancer or microbial infection within the subject.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The pharmaceutical compositions can be administered more than once a day, preferably once or twice a day.

In one embodiment of the present invention, therapeutically effective amounts of compounds of the present invention may range from approximately 0.05 to 50 mg per kilogram body weight of the recipient per day; preferably about 0.01-25 mg/kg/day, more preferably from about 0.5 to 10 mg/kg/day. Thus, for administration to a 70 kg person, the dosage range would most preferably be about 35-70 mg per day.

In another embodiment of the present invention, dosages may be estimated based on the results of experimental models, optionally in combination with the results of assays of the present invention. Generally, daily oral prophylactic doses of active compounds will be from about 0.01 mg/kg per day to 2000 mg/kg per day. Oral doses in the range of 10 to 500 mg/kg, in one or several administrations per day, may yield suitable results. In the event that the response of a particular subject is insufficient at such doses, even higher doses (or effective higher doses by a different, more localized delivery route) may be employed to the extent that patient tolerance permits. Multiple doses per day are also contemplated in some cases to achieve appropriate systemic levels of the composition.

Use of a long-term release implant may be particularly suitable in some cases.

“Long-term release,” as used herein, means that the implant is constructed and arranged to deliver therapeutic levels of the composition for at least 30 or 45 days, and preferably at least 60 or 90 days, or even longer in some cases. Long-term release implants are well known to those of ordinary skill in the art, and include some of the release systems described above.

Any suitable dosage may be administered. The type of microbial infection, or neoplasia to be treated, the compound, the carrier, and the amount will vary widely depending on body weight, the severity of the condition being treated and other factors that can be readily evaluated by those of skill in the art. Generally a dosage of between about 1 mg per kg of body weight and about 100 mg per kg of body weight is suitable.

A dosage unit may include a single compound of the present invention or mixtures thereof with other compounds or other anti-cancer agents, if the composition is used to treat cancer, or other antimicrobial agents, such as commonly used antibiotics such as vancomycin and streptomycin, if the composition is used to treat TB or another microbial infection. The dosage unit can also include diluents, extenders, carriers and the like. The unit may be in solid or gel form such as pills, tablets, capsules and the like or in liquid form suitable for oral, rectal, topical, intravenous injection or parenteral administration or injection into or around the tumor or localized site of microbial infection.

In pharmaceutical dosage forms, agents may be administered alone or with an appropriate association, as well as in combination, with other pharmaceutically active compounds. As used herein, “administered with” means that at least one pharmacological agent and at least one other adjuvant (including one or more other pharmacological agents) are administered at times sufficiently close that the results observed are indistinguishable from those achieved when one pharmacological agent and at least one other adjuvant (including one or more other pharmacological agents) are administered at the same point in time. The pharmacological agent and at least one other adjuvant may be administered simultaneously (i.e., concurrently) or sequentially. Simultaneous administration may be carried out by mixing at least one pharmacological agent and at least one other adjuvant prior to administration, or by administering the pharmacological agent and at least one other adjuvant at the same point in time. Such administration may be at different anatomic sites or using different routes of administration. The phrases “concurrent administration,” “administration in combination,” “simultaneous administration” or “administered simultaneously” may also be used interchangeably and mean that at least one pharmacological agent and at least one other adjuvant are administered at the same point in time or immediately following one another. In the latter case, the at least one pharmacological agent and at least one other adjuvant are administered at times sufficiently close that the results produced are synergistic and/or are indistinguishable from those achieved when the at least one pharmacological agent and at least one other adjuvant are administered at the same point in time. Alternatively, a pharmacological agent may be administered separately from the administration of an adjuvant, which may result in a synergistic effect or a separate effect. The methods and excipients described herein are merely exemplary and are in no way limiting.

The compounds of the present invention can be administered alone, in combination with each other, or they can be used in combination with other known compounds. For instance, the compounds can be used in conjunctive therapy with other known anti-angiogenic chemotherapeutic or antineoplastic agents (e.g., vinca alkaloids, antibiotics, antimetabolites, platinum coordination complexes, etc.). For instance, the compounds can be used in conjunctive therapy with a vinca alkaloid compound, such as vinblastine, vincristine, taxol, etc.; an antibiotic, such as adriamycin (doxorubicin), dactinomycin (actinomycin D), daunorubicin (daunomycin, rubidomycin), bleomycin, plicamycin (mithramycin) and mitomycin (mitomycin C), etc.; an antimetabolite, such as methotrexate, cytarabine (AraC), azauridine, azaribine, fluorodeoxyuridine, deoxycoformycin, mercaptopurine, etc.; or a platinum coordination complex, such as cisplatin (cis-DDP), carboplatin, etc. In addition, the compounds can be used in conjunctive therapy with other known anti-angiogenic chemotherapeutic or antineoplastic compounds. In pharmaceutical dosage forms, the compounds may be administered in the form of their pharmaceutically acceptable salts, or they may also be used alone or in appropriate association, as well as in combination with other pharmaceutically active compounds against TB, cancer and other microbial infections.

The specifications for the unit dosage forms of pharmacological agents of the present invention depend on, for example, the particular pharmacological agent(s) employed and the effect to be achieved, the pharmacodynamics associated with the particular pharmacological agent(s) in the subject, etc. Unit dosage forms for oral or rectal administration such as syrups, elixirs, and suspensions may be provided wherein each dosage unit, for example, teaspoonful, tablespoonful, tablet or suppository, contains a predetermined amount of a pharmacological agent. Similarly, unit dosage forms for injection or intravenous or other suitable administration route may include the pharmacological agent(s) in a composition as a solution in sterile water, normal saline or another pharmaceutically acceptable carrier.

Embodiments of the present invention include administering an effective amount of a first agent and an effective amount of a second agent. For example, embodiments may include administering a first agent and a second agent to provide an enhanced therapeutic effect. By “enhanced therapeutic effect” is meant that at least the desired outcome occurs more quickly and/or is of greater magnitude with a combination of the pharmacological agents, as compared to the same doses of each component given alone; or that doses of one or all component(s) are below what would otherwise be a minimum effective dose (a “sub-MED”).

Any two pharmacological compositions may be given in close enough temporal proximity to allow their individual therapeutic effects to overlap. For example, embodiments of the subject invention include the co-timely administration of a first and second agent, where “co-timely” is meant administration of a second pharmacological agent while a first pharmacological agent is still present in a subject in a therapeutically effective amount. It is to be understood that in some instances this will require sequential administration. Alternatively, multiple routes of administration may be employed, e.g., intravenous or subcutaneous injection combined with oral administration, and the like.

Embodiments also include pharmaceutical compositions in unit dosage forms that are useful which contain more than one type of pharmacological composition. In other words, a single agent administration entity may include two or more pharmacological agents. For example, a single tablet, capsule, dragee, trocheem suppository, syringe, and the like, combining two or more pharmacological agents would be a unit dosage form. The therapeutic agents present in a unit dosage form may be present in amounts such that, upon administration of one or more unit doses of the composition, a subject experiences, e.g., a longer lasting efficacy than with the administration of either agent alone and/or greater magnitude and/or quicker lowering of action. Such compositions may be included as part of a therapeutic package in which one or more unit doses are placed in a finished pharmaceutical container. Labeling may be included to provide directions for using the composition according to the invention. The actual amounts of each agent in such single unit dosage forms may vary according to the specific compositions being utilized, the particular compositions formulated, the mode of application, the particular route of administration, and the like, where dosages for a given subject may be determined using conventional considerations, e.g., by customary comparison of the differential activities of the subject compositions and of a known agent, or by means of an appropriate, conventional pharmacological protocol.

Therapeutically effective dosages for the compounds described herein can be estimated initially from cell culture assays. For example, a dose can be formulated in animal models to achieve a circulating concentration range that includes the IC₅₀ as determined in cell culture (i.e., the concentration of test compound that is lethal to 50% of a cell culture), or the IC₁₀₀ as determined in cell culture (i.e., the concentration of compound that is lethal to 100% of a cell culture). Such information can be used to more accurately determine useful doses in humans. Initial dosages can also be estimated from in vivo data.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: “The Pharmacological Basis of Therapeutics”, Ch. 1, p. 1).

Dosage amount and interval may be adjusted individually to provide plasma levels of the active compound which are sufficient to maintain therapeutic effect. Preferably, therapeutically effective serum levels will be achieved by administering multiple doses each day. In cases of local administration or selective uptake,—the effective local concentration of the drug may not be related to plasma concentration. One having skill in the art will be able to optimize therapeutically effective local dosages without undue experimentation.

In one embodiment, a catheter is used to direct the composition directly to the location of the targeted tumor or microbial infection. As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired affects and the therapeutic indication. Typically, dosages may be between about 10 μg/kg and 100 mg/kg body weight, preferably between about 100 μg/kg and 10 mg/kg body weight. Alternatively dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g., et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be vary dependent upon the percent composition of the dosage of compounds of the present invention, in most cases some generalizations regarding the dosage can be made. The daily dosage regimen for an adult human patient may be, for example, an oral dose of between 0.1 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the compounds disclosed herein in amounts that exceed, or even far exceed, the above-stated dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

An additional embodiment of the present invention is the use of the above described syringopeptides and rhamnolipids to inhibit or kill microbial cells (microorganisms). The microorganisms may be bacterial cells, fungal cells, protozoa, viruses, or eukaryotic cells infected with pathogenic microorganisms. The method generally is directed towards the contacting of microorganisms with the syringopeptide. The contacting step can be performed in vivo, in vitro, topically, orally, transdermally, systemically, or by any other method known to those of skill in the art. The contacting step is preferably performed at a concentration sufficient to inhibit or kill the microorganisms. The concentration of the syringopeptide can be at least about 0.1 μM, at least about 0.5 μM, at least about 1 μM, at least about 10 μM, at least about 20 μM, at least about 50 μM, or at least about 100 μM. The methods of use can be directed towards the inhibition or killing of microorganisms such as bacteria, gram positive bacteria, gram negative bacteria, mycobacteria, yeast, fungus, algae, protozoa, viruses, and intracellular organisms. Specific examples include, but are not limited to, Staphylococcus, Staphylococcus aureus, Pseudomonas, Pseudomonas aeruginosa, Escherichia coli, Chlamydia, Candida albicans, Saccharomyces, Saccharomyces cerevisiae, Schizosaccharomyces pombe, Trypanosoma cruzi, or Plasmodium falciparum. The contacting step can be performed by systemic injection, oral, subcutaneous, IP, IM, IV injection, or by topical application. For injection, the dosage can be between any of the following concentrations: about 1 mg/kg, about 5 mg/kg, about 10 mg/kg, about 25 mg/kg, about 50 mg/kg, about 75 mg/kg, and about 100 mg/kg. The contacting step can be performed on a mammal, a cat, a dog, a cow, a horse, a pig, a bird, a chicken, a plant, a fish, or a human.

In one embodiment, syringopeptides for antibacterial applications can include a polypeptide having the sequence shown in SEQ ID NO:1, and SEQ ID NO:2.

In one embodiment, syringopeptides for antifungal applications can include a polypeptide having the sequence shown in SEQ ID NO:1, and SEQ ID NO:2.

An additional embodiment of the invention is the use of any of the above described syringopeptides to inhibit or kill cancer cells. The method generally is directed towards the contacting of cancer cells with the syringopeptide. The contacting step can be performed in vivo, in vitro, topically, orally, transdermally, systemically, or by any other method known to those of skill in the art. The contacting step is preferably performed at a concentration sufficient to inhibit or kill the cancer cells. The concentration of the syringopeptide can be at least about at least about 0.1 μM, at least about 0.5 M, at least about 1 μM, at least about 10 μM, at least about 20 μM, at least about 50 μM, or at least about 100 μM. The cancer cells can generally be any type of cancer cells. The cancer cells can be sarcomas, lymphomas, carcinomas, leukemias, breast cancer cells, colon cancer cells, skin cancer cells, ovarian cancer cells, cervical cancer cells, testicular cancer cells, lung cancer cells, prostate cancer cells, and skin cancer cells. The contacting step can be performed by subcutaneous, IP injection, IM injection, IV injection, direct tumor injection, or topical application. For injection, the dosage can be between any of the following concentrations: about 0.1 mg/kg, about 1 mg/kg, about 5 mg/kg, about 10 mg/kg, about 25 mg/kg, about 50 mg/kg, about 75 mg/kg, and about 100 mg/kg. The contacting step can be performed on a mammal, a cat, a dog, a cow, a horse, a pig, a bird, a chicken, a plant, a fish, a goat, a sheep, or a human. The inhibition of cancer cells can generally be any inhibition of growth of the cancer cells as compared to the cancer cells without syringopeptide treatment. The inhibition is preferably at least about 10%, 20%, 30%, 40%, 50%, 60%, 70%, 80%, 90%, 95%, 96%, 97%, 98%, 99%, and ideally 100% inhibition of growth. The inhibition may be achieved by lysis of the cancer cells or by other means. The cancer inhibiting syringopeptide can be used synergistically with other cancer chemotherapeutic agents.

In one embodiment, syringopeptides for anticancer applications can include a polypeptide having the sequence shown in SEQ ID NO:1, and SEQ ID NO:2.

A further embodiment of the invention is directed towards methods for the additive or synergistic enhancement of the activity of a therapeutic agent. The method can comprise preparing a composition, wherein the composition comprises a syringopeptide and a therapeutic agent. Alternatively, the method may comprise co-therapy treatment with a syringopeptide (or syringopeptides) and/or rhamnolipids used in conjunction with other therapeutic agents. The syringopeptide can be any of the above described syringopeptides. The therapeutic agent can generally be any therapeutic agent, and preferably is an antibiotic, an antimicrobial agent, a growth factor, a chemotherapy agent, an antimicrobial agent, lysozyme, a chelating agent, or EDTA. Preferably, the activity of the composition is higher than the activity of the same composition containing the therapeutic agent but lacking the syringopeptide. The composition or co-therapy can be used in in vitro, in vivo, topical, oral, IV, IM, IP, and transdermal applications. The enhancement of the activity of the composition containing the therapeutic agent and the syringopeptide is preferably at least 10%, 20%, 30%, 40%, 50%, 60%, 70%, 80%, 90%, 100%, 125%, 150%, 175%, or 200% relative to the activity of the therapeutic agent alone.

Generally, any syringopeptide which is active on a stand-alone basis against a target can be used to increase either additively or synergistically the activity of another therapeutic agent against that target. If several syringopeptides are candidates for a given synergy application, then the less toxic syringopeptides would be more favorably considered.

The following examples are included to demonstrate embodiments of the invention. It should be appreciated by those of skill in the art that the techniques disclosed in the examples which follow represent techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### VII. Organic Synthesis of RLs and SPs

The compounds disclosed herein may also be synthesized by methods described below, or by modification of these methods. Ways of modifying the methodology include, among others, temperature, solvent, reagents etc., and will be obvious to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and Greene & Wuts, Protective Groups in Organic Synthesis, John Wiley & Sons, 1991, which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety.

The following preparations and examples are given to enable those skilled in the art to more clearly understand and to practice the present invention. They should not be considered as limiting the scope of the invention, but merely as being illustrative and representative thereof.

The starting materials and reagents used in preparing these compounds are either available from commercial suppliers such as Aldrich Chemical Co., (Milwaukee, Wis., USA), Bachem (Torrance, Calif. USA), Emka-Chemie, or Sigma (St. Louis, Mo., USA) or are prepared by methods known to those skilled in the art following procedures set forth in references such as Fieser and Fieser's Reagents for Organic Synthesis, Volumes 1-15 (John Wiley and Sons, 1991); Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (John Wiley and Sons, 5th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

A. Rhamnolipids

In general, rhamnolipids of FIG. 2 may either be isolated from their natural source or synthesized through the construction of a synthetic rhamnolipid library. The rhamnolipids library represents variations in the amphiphilic character of the molecules by introducing specific modifications both in the fatty acid part and in the sugar component. This is achieved particularly through variation in the number, lengths, and stereochemistry of the β-hydroxylated carboxylic acids and in the number of sugars, and also through reduction of the free carboxylate to an uncharged alcohol moiety.

Achiral β-ketoesters of different lengths are generated by C-acylation of Meldrum's acid. Asymmetric reduction of the ketones in the presence of a chiral ruthenium 2,2′-bis(diphenylphosphino)-1,1-binaphthyl (BINAP) catalyst yielded the enantiomerically pure β-hydroxy esters. These secondary alcohols are obtained in high yields and with optical purities, as determined by NMR spectroscopy and chiral GC separation of the corresponding Mosher's ester derivatives. Subsequent saponification and silylation furnished the corresponding 3-O-triethylsilyl-substituted carboxylic acids as optically pure key building blocks. For the synthesis of rhamnolipid alcohols, a terminally protected 1,3-diol building block is required. This may be accessed via an appropriately chosen 3-(2-methoxyethoxy)methyl-protected acetate.

After esterification, C₁₈ reversed-phase silica support (RP-18) is added to the reaction mixture, the solvents are evaporated, and all monolipid starting materials are easily and quantitatively removed by filtration with MeOH/water. Subsequently, the silylated esters are deprotected with dilute trifluoroacetic acid (TFA) while attached to the solid support. After washing and filtration, MgSO₄ is added and the diastereomerically pure 3-hydroxy dilipid compounds are desorbed quantitatively with CH₂Cl₂. The pure products obtained from the previous solid-phase step are glycosylated with an excess of a rhamnose donor in CH₂Cl₂. The progress of the reaction is monitored by TLC and MALDI-MS. Workup of the resulting compounds is performed by a phase switch from solution phase back to the RP-18 solid support. Removal of the temporary phenoxyacetate (POAc) protecting group at the 2-position of rhamnose is effected on the solid support, as is the final removal of the butane-2,3-dione (BDA) protective group. For the construction of higher glycosylated rhamnolipid methyl esters, the resulting compounds of the previous synthetic step are glycosylated in a second or third hydrophobically assisted switching phase (“HASP”) reaction cycle, yielding pure products without the need for a single purification step.

Cleavage of the methyl ester group is most successful under enzymatic conditions. Therefore, a solid-supported lipase from Candida antarctica is used which furnishes the rhamnolipid acids in good to excellent yields and with exceptionally broad substrate tolerance. All the (R,R′)-configured rhamnolipid methyl esters are hydrolyzed to the corresponding diastereomeric RL-acids, however, the (3S)-configured fatty acid methyl esters sometimes resist successful substrate recognition by the enzyme. Therefore, lipid esters amenable to chemical deprotection are synthesized and employed for HASP construction of the corresponding RL esters. Because the trichloroethyl ester undergoes transesterification upon treatment with MeNH₂ in MeOH, the benzyl ester is employed to access (R,S′)-configured rhamnolipid acids. Excellent yields of both (R,S′)-RL acids and rhamnolipid alcohols are accomplished through analogous HASP cycles.

B. Syringopeptins

The synthetic strategies that follow are generally related to the synthesis of peptides, and are known to those having ordinary skill in the art of peptide synthesis. In general, syringopeptins of FIG. 1 may be synthesized according to the following general strategy for the synthesis of cyclic lipononadepsipeptides utilizing the principles of solid-phase peptide synthesis. The strategy is based on the use of a mild orthogonal protection scheme and the incorporation of the non-proteinogenic amino acid (Z)-2,3-didehydro-2-aminobutyric acid) (“Dhb”) into the peptide chain as the dipeptide Fmoc-Thr(tBu)-(Z)-Dhb-OH. The didehydrodipeptide is synthesized by a water soluble carbodiimide inducing beta-elimination of a protected dipeptide containing a residue of Thr with its free hydroxyl side chain unprotected.

First, Alloc-Ser-OH and the didehydropeptide Fmoc-Thr(tBu)-(Z)-Dhb-OH have to be synthesized. Alloc-Ser-OH may be prepared by using the trimethylsilyl (TMS) group as a temporary protecting group for all functional positions to avoid the undesired formation of side products such as di- and tripeptides. Thus, for example, Ser may be treated with trimethylsilyl chloride in CH₂Cl₂ in the presence of N,N-diisopropylethylamine (“DIEA”), and the intermediate product would then be treated in situ with allyl chloroformate. Finally, removal of the remaining protecting TMS groups would occur upon aqueous workup to yield the final product.

Synthesis of syringopeptins of the present invention may utilize 1-ethyl-3-(3-dimethylaminopropyl) carbodiimide hydrochloride as an activating reagent for the hydroxyl function in the presence of CuCl in CH₂Cl₂/N,N-dimethylformamide (DMF) under a nitrogen atmosphere.

Removal of the allyl group can be performed with [Pd(PPh₃)₄] in the presence of PhSiH₃ under argon. The resulting target dipeptide would then be ready to be incorporated into the peptide sequence. Stereoselectivity for these reactions is generally excellent.

As an example of the incorporation of Fmoc protected amino acids into the peptide chain of syringopeptins of the present invention, the incorporation of Fmoc-Gly-OH onto Barlos resin may be performed in the presence of DIEA. Peptide chain elongation may be carried out by using an Fmoc/tBu strategy. Removal of the Fmoc group can be achieved with piperidine/DMF. The majority of subsequent couplings can be performed with N,N-diisopropylcarbodiimide (DIPCDI)/1-hydroxybenzotriazole (HOBt) as the coupling reagent; however, the incorporation of the third amino acid (Alloc-Ser-OH), Fmoc-Thr(tBu)-OH, and the dipeptide Fmoc-Thr(tBu)-(Z)-Dhb-OH could be carried out by following other strategies. Thus, Alloc-Ser-OH is incorporated using N-[(1H-benzotriazol-1-yl)(dimethylamino)methylene]-N-methylmethanaminium tetrafluoroborate N-oxide (TBTU)/DIEA, a very powerful coupling reagent. The addition of Fmoc-Thr(tBu)-OH to form the ester linkage could be carried out using DIPCDI and a catalytic amount of 4-dimethylaminopyridine (DMAP). The carboxyl function of Fmoc-Thr(tBu)-(Z)-Dhb-OH can often times be less reactive than the corresponding group in carbamate-protected amino acids. Therefore, 1-hydroxy-7-azabenzotriazole (HOAt) can be used instead of HOBt. The Alloc group may be removed by using [Pd(PPh₃)₄] in the presence of PhSiH₃ under argon.

The cleavage of the protected peptide from the resin can be carried out smoothly with trifluoroacetic acid (TFA)/CH₂Cl₂ and the crude product may be evaluated by reversed-phase HPLC or other spectroscopic methods. The cyclization step can be carried out in CH₂Cl₂ with DIPCDI/HOBt/DIEA. DIEA may be added to neutralize the trifluoroacetate salt and avoid undesired trifluoroacetylation. As with many synthetic schemes involving solid phase peptide synthesis, the protected cyclic peptide is treated with TFA/H₂O in order to purifying the final peptide. However, the unprotected final cyclic peptide can often be extremely insoluble and often precludes the purification of the peptide by the aforementioned method, so other commonly used purification methods must be used, such as reverse phase column chromatography followed by the removal of the tBu-based protecting groups with TFA/H₂O. The final syringopeptin can then be obtained by washing the crude product with Et₂O.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the fall scope of equivalents to which such claims are entitled.

All patents, patent applications and publications cited in this application are hereby incorporated by reference in their entirety for all purposes to the same extent as if each individual patent, patent application or publication were so individually denoted.

### EXAMPLES

The following examples illustrate specific embodiments of the invention, but are not intended to limit the scope of the invention in any way.

### Example 1

### Purification of RLs and SP 25A

SP 25A was purified from Pseudomonas syringae pv. syringae as described by Bidwai et al. (Plant Physiology, 1987, 83:39-43).

Briefly, a culture was grown for 10 d to stationary phase at room temperature (˜25° C.). The cells were spun down in a centrifuge and the supernatant from the growth media was then extracted with acidified acetone, concentrated with a rotary evaporator, purified to homogeneity by reverse phase HPLC, and lyophilized for storage at 4° C. until further use. The purity and molecular weight of the isolated compound were verified by MALDI-TOF analysis at the Center for Integrated BioSystems (Logan, Utah) and matched known spectra of SP 25A.

Commercial RL samples were obtained as a 25.1% aqueous solution (product JBR-425; Lot#021004) from Jeneil Biotech, Inc. (Saukville, Wis.). The purity and molecular weight of the RLs were determined using MALDI-TOF at the Center for Integrated BioSystems. The relative concentrations of the two different rhamnolipid moieties (Decanoic acid, 3-[(6-deoxy-L-mannopyranosyl) oxy]-1-(carboxymethyl) octyl ester, and Decanoic acid, 3-[(6-deoxy-2-O (6-deoxy-L-mannopyranosyl)-L-mannopyranosyl]oxy]-1-(carboxymethyl)octyl ester) in the commercial rhamnolipid mixture were determined by ¹³C NMR.

After purification, SP 25A was subjected to MALDI-TOF and HPLC analysis to confirm the purity of the fractionated compound. HPLC analysis revealed a single peak, as did MALDI TOF. This single major peak had a molecular weight of 2,400.37 Da, which was in agreement with the reported mass of SP 25A.

The commercial RL preparation was subjected to MALDI-TOF analysis and ¹³C NMR to determine the relative concentration and isoform content of the mixture. Mass spectrum analysis revealed Rhamnose-C10-C10 (MW=503.31) and Rhamnose-Rhamnose-C10-C10 (MW=649.33), in agreement with the product data sheet. Analysis of the ¹³C NMR spectra revealed that the isoforms were present in an equimolar ratio.

### Example 2

### Inhibition of Microbial Growth by RLs and SP 25A

Listeria monocytogenes EGDe was thawed and subcultured twice at 37° C. in brain heart infusion broth (BHI) (DIFCO, Franklin Lanes, N.J.). An overnight culture was diluted 10 fold in sterile medium and grown for 4 h to an OD₆₀₀ of 1.7, which corresponded to ˜10⁹ cfu/mL. The cell preparation was exposed to either 3 μg/mL SP 25A or 6 μg/mL RLs. Treatment with both RLs and SP 25A caused membrane permeabilization (FIG. 6A), but each reduced cell growth (FIG. 6B) of L. monocytogenes by different amounts. The addition of RLs resulted in more membrane permeabilization than did the addition of SP 25A. After 30 min of incubation with RLs, the permeabilization increased by 53%, while permeabilization due to SP 25A increased merely 2.6% during the same time. Unexpectedly, the permeabilization declined after 120 min of treatment with RLs.

Cell density was highest in the control, and lowest with the addition of SP 25A. The control culture grew by 15.3% during 120 min, while the culture treated with RLs grew by 8%, cultures treated with SP 25A did not grow. The membrane permeabilization and the cell density changes did not have a positive correlation (see FIGS. 6A-6B). This lack of positive correlation suggests that the mechanism of action for both compounds is not only due to membrane permeabilization.

### Example 3

### Rate of Antimicrobial Action and MICs for RLs and SP 25A

The antimicrobial action for each compound was initially determined by the rate of uptake of propidium iodide (Fluoropure grade, Molecular Probes, Inc., Eugene, Oreg.) as previously described (Haughland, R. P., 2002, Handbook of fluorescent probes and research chemicals, 9th ed. Molecular Probes, Inc., Eugene, Oreg.). Briefly, all cultures were grown overnight in their respective optimal growth medium and temperature from freezer vials (Table 1). Each culture was sub-cultured twice, harvested in mid-log phase, washed with saline, and adjusted to an appropriate concentration by measuring the optical density at 600 nm. PI, with an excitation wavelength of 535 and an emission wavelength of 617, was added to the culture suspension at a final concentration of 10 μM. Each organism was treated with 50 μg/mL SP 25A and 60 μg/mL of the RL mixture in a final volume of 2.2 mL. The increase in fluorescence was measured with a Shimadzu RF 1501 spectrophotofluorometer at 15 s intervals for a maximum period of 120 min. Saline was added in place of SP 25A or RLs as a negative control. All inhibition experiments were done in replicate.

The rate of antimicrobial action was expressed as the inhibition rate (“IR”). Curve fitting was done using OriginPro version 7.0 (Natick, Mass.). IR=((Log RFU/(Time))−C)/Time (when d Log RFU/dT>0). Where RFU=relative fluorescent units; and C=Y intercept.

The MIC of the compounds for the organisms was determined by the microbroth dilution method, described hereafter. The microorganisms were prepared as described above and resuspended in their optimal growth media (Table 1) to ˜10⁵ CFU/mL containing SP 25A at 2, 3, 4, 5, 6, 7, 8, 16, 32 and 50 μg/mL in a total volume of 550 μL. RL concentrations of 2, 3, 4, 5, 6, 7, 8, 16, 32 and 60 μg/mL in a total volume of 550 μL were tested in a 48-well plate (Corning, N.Y.). The plates were incubated at optimal growth conditions for the respective organism and monitored for an increase in OD₆₀₀ after 48 h by a Perkin-Elmer (HTS 7000) plate reader (Downers Grove, Ill.). A positive control (inhibition of growth) using Polymyxin B (Sigma-Aldrich Cat# P0972) at 1000 μg/mL for all gram-negative organisms, Penicillin G (Sigma-Aldrich Cat# P3032) at 1000 μg/mL for the gram-positive organisms, Rifampicin (Sigma-Aldrich Cat# 3501) at 1000 μg/mL was used for M. smegmatis, E. faecalis and S. aureus. Negative controls (no inhibition of growth) were included using saline in the assay for each compound. The lowest concentration at which there was no increase in OD over 48 h was reported as the MIC. Each MIC was determined in replicate with three separate tests per replication. The results of the three tests were averaged for each replicate.

Synergistic activity between SP 25A and RLs was measured by exposing L. monocytogenes to RLs at a concentration of 0, 0.5, 1, 1.5, 3, and 6 μg/mL alone and in combination with 3 μg/mL SP 25A and monitoring PI uptake as previously described. The experiment was done in replicate and repeated.

M. smegmatis was used as a surrogate organism for M. tuberculosis. In the present study, we determined that SP 25A inhibited M. smegmatis at 4 μg/mL.

We determined that RLs were active against multiple strains of gram-positive bacteria but effective against only one gram-negative bacterium (F. devorans) at <60 μg/mL. We also observed that RLs inhibited bacterial spore germination in B. subtilis and C. sporogenes at 4 μg/mL.

Since both compounds demonstrated a similar range of activity and MICs, we investigated if either of the individual MIC of RLs or SP 25A would be reduced when both of the compounds were used at the same time. This was done by exposing L. monocytogenes to mixtures of SP 25A at 3 μg/mL with various RLs concentrations. The IR for the mixture of both the compounds was significantly different (p<0.05) than the IR of the compounds used alone across all concentrations tested. We achieved a higher rate of antimicrobial activity when both the compounds were used in combination as compared to individual use. Using the compounds together, we were able to achieve the same level of inhibition with up to 6-fold less of a concentration of RLs. The increase in antibiotic effectiveness followed a sigmoidal curve (FIG. 5).

The present study is the first to define the synergistic antibacterial activity from using a combination of both SP 25A and RLs.

### Example 4

### Determination of Toxicity of SP 25A and RLs in Mammalian Cells

Toxicity of the two compounds to mammalian cells was assayed in cell culture using mouse enteroendocrine cells (STC-1), human embryonic kidney cells (HEK 293; ATCC CRL-1573), and human lung fibroblasts (LL47; ATCC CCL-135). Each cell line was subjected to SP 25A and RLs at the respective MIC (e.g., 4 μg/mL and 8 μg/mL). The human embryonic kidney cells and human lung fibroblasts were grown as per the American Type Culture Collection's recommendation, while the mouse enteroendocrine cells were grown as described by Vincent et al. (2001, Proc. Natl. Acad. Sci., 99:2392-2397). Media and sera were purchased from HyClone Laboratories (Logan, Utah). All cells were grown in 10% fetal bovine serum (FBS).

FIG. 5 includes graphs that illustrate the toxicity of RLs and SP 25A against cell cultures (e.g., STC (panel A), HEK 293 (panel B) and LL-47 (panel C)). The number in the parenthesis represents the concentration of each compound in μg/mL. The percent cell death was benchmarked to 100% lysis with the positive control. The number of total cells and dead cells were counted after 6, 24, and 48 h using a Nucleocounter Automated cell counting system (New Brunswick; Edison, N.J.). Briefly, cells (STC, 200,000 cells/well; HEK 293, 200,000 cells/well; and LL47, 100,000 cells/well) were incubated in the appropriate medium for 24 h prior to addition of fresh media containing the antimicrobial compounds. After addition of the antimicrobial compound, the cell cultures were incubated at 37° C. with 5% CO₂ for 6, 24, and 48 h. Cells were harvested by trypsinization using 0.25% trypsin-EDTA for 2 min. The trypsin was neutralized by addition of 200 μL of serum containing fresh medium. The cells were then harvested and transferred to 1.5 mL tubes, centrifuged (3-5 min at <100×g), and resuspended in 200 μL of fresh medium. For the total cell count, 100 μL of the suspension was added to lysis buffer (Reagent A100 in the starting kit (Cat No. M1293-0020, New Brunswick Scientific) for 30 s, which was stabilized using 100 μL of Reagent B. A positive control of completely lysed cells was used along with a negative control using sterile PBS (pH 7.4). For dead cell counts, 100 μL of cell lysate was counted without the use of lysis buffer or stabilizing buffer. All cell counts were obtained using the Nucleocounter automated cell counting system. Data were reported as the percent of cell death. The toxicity testing was done in replicate using three wells per test.

It is thought that SP 25A and RLs target the cell membrane, and are effective as antibiotics through inducing lysis of the effected microbes. This study used PI as a probe to monitor cell membrane integrity during cellular exposure to both RLs and SP 25A. PI accumulation directly correlated to increasing exposure time for each compound, indicating that each of the compounds compromised the cell membrane. As such, the rate of PI accumulation was used to compare the inhibitory rate for each organism tested (see FIG. 3). FIG. 3 shows the inhibition rate for SP 25A at 50 μg/mL and the inhibition rate for RLs at 60 μg/mL for various microorganisms.

SP 25A inhibited all gram-positive organisms tested. However, SP 25A did not inhibit the growth of any of the gram-negative organisms tested except F. devorans. Additionally, SP 25A did not inhibit the growth of any strain of yeast that was tested including, Brettonomyces bruxellensis, Candida vini, Pichia fermentans, Saccharomyces luduigi, Metschinikowia puicherrima, and Kloeckera apiculata (data not shown). The highest rate of inhibition was found for Brevibacterium linens, while E. faecalis had the lowest rate of inhibition (FIG. 3).

As observed with SP 25A, RLs inhibited only gram-positive bacteria (with the exception of F. devorans), with the rate of inhibition being the fastest against B. subtilis (FIG. 3) and slowest against both the Listeria species that were tested. The rate of inhibition for each tested species of bacteria was different for RLs and SP 25A, however, the distribution of bacterial species that were inhibited by RLs was the same as those that were inhibited by SP 25A.

There was a significant difference (p<0.01) in the rate of PI accumulation between SP 25A and RLs. Depending upon the species, RLs were 3 to 433 times faster in compromising the cell membrane as compared to SP 25A, the difference being highest for enterococci and lowest being for the Listeria species

The MIC for each compound was determined for each bacterial species (Table 2). The SP 25A MIC ranged from 3 to 16 μg/mL, while the MICs for RLs ranged from 4 to 32 μg/mL for the organisms tested. A substantial difference in the MIC for SP 25A and RLs in E. faecalis and S. aureus was observed (Table 2). While the MIC for RLs in both these organisms was >60 μg/mL, SP 25A completely inhibited growth of both organisms at 8 μg/mL. For all the other organisms, SP 25A had a similar or lower MIC as compared to RLs. SP 25A and RLs inhibited growth of M. smegmatis at 4 μg/mL. Both compounds inhibited spore germination from Bacillus and Clostridium at 4 μg/mL. This work is the first report of anti-spore activity by these compounds.

Three mammalian cell lines were used to assess cytotoxic effects for each compound at 4 μg/mL and 8 μg/mL. No significant (p>0.05) cytotoxicity was observed at 6, 24, and 48 h after exposure to each compound at either of the concentrations (FIG. 5). While a small amount of lysis was observed, it was not above background. Cells treated with triton (positive control) showed 100% lysis. These observations indicate that neither compound completely compromised the host membrane. Other groups have reported haemolytic activity for SP 25A. Dalla Serra et al (Dalla Serra, M., I. Bernhart, P. Nordera, D. Di Giorgio, A. Ballio, and G. Menestrina, 1999, Mol. Plant. Microbe Interact. 12:401-9) reported a value of 8.88 μg/mL of SP 25A to achieve 50% RBC hemolysis. In contrast, we did not observe membrane permeabilization of any of the three cell lines tested while using commensurate concentrations of RLs and SP 25A. A possible explanation of this observation is that RBC's lack an endomembrane, which is thought to play a central role in the rapid resealing response in event of plasma membrane disruption.

### Example 5

### Genetic Profiling

Total RNA was extracted from a 1.8 mL culture immediately before treating the cells with SP 25A and RLs (T₀), after 30 min (T₃₀), and at 120 min (T₁₂₀) of exposure at 37° C. Simultaneously, the cell density was measured at OD₆₀₀. Membrane permeabilization was determined by measuring the PI uptake. Total RNA extraction and reverse transcription (from 10 μg total RNA) was done as described by Xie Y. et al. (2004, Appl. Environ. Microbiol. 70:6738-6747) to produce biotinylated cDNA, which was sheared with DNase1 as described by the protocol of NimbleGen Systems (Madison, Wis.). The optimized NimbleScreen chip that was used contained 12 wells, enabling the entire experiment to be done on a single chip. Each well contained five probes for each open reading frame in the entire genome.

Hybridization of the fluorescently-labeled (Cy3-streptavidin; Amersham Biosciences, Piscataway, N.J.) cDNA (500 ng) was done using a custom NimbleScreen chip optimized for L. monocytogenes EGDe, as described by the NimbleGen Systems protocol. Hybridization was detected with a Genepix 4200A array scanner (Axon Instruments, Union City, Calif.) at the Center for Integrated BioSystems. Data extraction from the scanned images was completed at NimbleGen Systems. The raw expression data from the entire experiment were normalized together using R with the robust multichip average (RMA) method. Annotations for Listeria monocytogens EGDe were obtained from the ERGO database (Integrated Genomics, Chicago, Ill.).

### Example 6

### Statistical Analysis and Data Visualization

RMA normalized data were analyzed using SAM Version 2.01 (Tusher, V. G., et. al. 2001, Proc. Natl. Acad. Sci., 98:5116-5121) with a one class time course experimental design using the xCluster R module (Center for Integrated BioSystems). Any gene with at least a log₂ ratio of ±0.58, which is equivalent to a 1.5 fold change, and a Q<0.3 was considered significant. The entire biological experiment was repeated twice. The log₂ ratios were calculated by taking a difference in log₂ intensity of a single time point with the preceding time point.

### Example 7

### Genes Differentially Regulated Upon Treatment with RLs

The expression data were examined for genes that were constitutively expressed above the mean expression level, but none were found, indicating that gene expression changed over the exposure time. At T₃₀, RLs induced eight genes and repressed two genes. Treating the cells for 120 min with RLs significantly altered the expression of 39 genes. Regulation of 21 common genes was observed between SP 25A and RLs (Table 3, which shows functional categories that contained the genes that were significantly differentially expressed in response to treatment with sub-MIC doses of RLs). Despite regulating these common genes, the patterns of expression of were different as a result of exposure to RLs and SP 25A.

RLs induced three PEP/PTS components, α-mannosidase (LMO0401), and five genes involved in glycolysis and the pentose phosphate pathway (Table 4, which shows significantly differentially regulated genes during exposure to RLs). Conversely, all these genes were repressed when cells were treated with SP 25A during the same time period. The H⁺-transporting ATP synthase C (atpE) was induced at T₃₀, but subsequently repressed at T₁₂₀.

No genes related to transcription were differentially regulated after treatment with RLs. Only one gene related to protein biosynthesis, phenylalanyl-tRNA synthetase alpha chain (pheS (LMO1221)) was induced at T₃₀. An acetyltransferase (LMO0624) involved in post translation modification was repressed at T₃₀, but induced at T₁₂₀ (Table 4).

Of the four virulence factors in the genome, only listeriolysin O (hly) was induced after 120 min of treatment. Expression of the remaining virulence factors was not changed by addition of RLs.

Four stress-related genes, single strand binding protein (ssb), non-heme iron binding ferritin (fri), heat shock protein (cspL), and peroxide operon regulator (perR) (LMO1683), were induced after 120 min. However, none of the genes in the perR regulon were induced. No other genes were significantly regulated during treatment with RLs.

### Example 8

### Genes Differentially Regulated Upon Treatment with SP 25A

Treating L. monocytogenes with SP 25A significantly altered the transcript profile of ˜5% of the genes of its genome. Addition of SP 25A to the growth medium repressed 97% of the 139 differentially regulated genes (Table 5, which shows functional categories that contained the genes that were significantly differentially expressed in response to treatment with sub-MIC doses of SP 25A). The gene profiling data generated were also analyzed for genes that were constitutively expressed above the mean level during the treatment in an effort to find genes that may be essential for survival of bacteria under the antimicrobial stress. No genes were found that were constitutively expressed above the mean. Most functional categories contained genes that were repressed. No categories contained genes that were only induced. However, a few categories (ABC transporters, carbohydrate metabolism, transcription regulators, secretion, virulence factors, and unknown genes) contained genes that were induced and repressed.

Four genes involved in cell division and chromosome replication were repressed. Genes encoding for cell division initiation protein, DivIVA (LMO1888), ATPase associated with chromosome architecture/replication (LMO2759), DNA gyrase subunit B (gyrB), and DNA gyrase subunit A (gyrA) were repressed with addition of SP 25A. The transcription factor lytR which is correlated to the decrease in activity of autolytic enzymes, was also repressed (Table 6, which shows significantly differentially regulated genes during exposure to SP 25A).

PEP/PTS transporters specific for β-glucosides, fructose, and trehalose; α-mannosidase (a sugar hydrolase); and 22 other genes in carbohydrate metabolism were differentially expressed during the treatment time. From the entire set of genes in the intermediary metabolism category, only L-glutamine-fructose-6-phosphate transaminase (LMO0726) and 6-phospho-β-glucosidase (LMO0739) were induced at T₃₀. However, at T₁₂₀ all of the 26 genes in sugar transport and intermediary metabolism were repressed (Table 6). Each of the PEP/PTS components was repressed after 120 min (Table 6).

In addition to the sugar transporters and ATPases that were repressed, the large-conductance mechanosensitive ion channel (LMO2064) was induced at T₃₀, but repressed at T₁₂₀. This mechanoreceptor is involved in osmoregulation. Other studies using gene expression profiling did not observe an expression change in this ion channel, despite its importance in restoring the osmotic stability in a cell. This observation may be indicative of membrane perturbation early in the treatment time.

The repressed genes in central intermediary metabolism included genes involved in glycolysis, 6-phospho-beta-glucosidase (LMO0739), the pyruvate dehydrogenase operon (pdhA, pdhB, pdhC, pdhD), lactate dehydrogenase (ldh), pyruvate kinase (pykA), and phosphoglyceromutase (LMO2205). Repression of genes in the pentose phosphate pathway was also observed, ribose 5-phosphate isomerase (LMO0736), transaldolase (LMO2743), ribulose 5-phosphate 3-epimerase (LMO0735, LMO2659), and fructose-1,6-bisphosphate aldolase (fbaA). The dihydroxyacetone kinase enzyme complex (LMO2695, LMO2696 and LMO2697), which is responsible for phosphorylation of dihydroxyacetone and glycerol prior to entry into the glycolytic pathway, was also repressed. Four out of six genes involved in Fe-S cluster biosynthesis (sufD, IscU, sufB, cysteine desulfurase (LMO2413)) were repressed.

Repression of key genes for cellular respiration was observed. After 120 min, two genes (out of eight) of the H⁺ transporting ATP synthase enzyme complex, which code for the alpha and c subunits (atpA and atpE), were repressed. Three of the four subunits for quinol oxidase (LMO0014, LMO0015, LMO0016) were also repressed.

At T₃₀ two genes involved in protein biosynthesis, (LMO2511 and rpsU) were induced. The gene LMO2511 codes for the ribosome associated factor Y, which is a global translation inhibitor, while rpsU codes for the S21 protein in the 30s ribosomal complex. After 120 min of treatment with SP 25A, three of the four subunits of RNA polymerase (rpoA, rpoB, rpoC) were repressed. After 120 min 11 ribosomal proteins (out of 59) were repressed and two elongation factors (out of total four) were repressed. Hence, after 120 min genes needed for transcription and translation were repressed.

At T₃₀ two of the virulence genes hly (listeriolysin O precursor) and fibronectin binding protein (LMO0727) were induced, while iap (an invasion associated protein) was repressed. At T₁₂₀, phospholipase C (plcA) was also repressed. Hence, after 120 min SP 25A led to the repression of four genes directly required for host invasion by L. monocytogenes.

At T₃₀ five stress-related genes were repressed, while four genes were induced. Among the repressed genes were two chaperone proteins (groEL, grpE), three oxidative stress genes (sod, msrA, trxB), and one gene related to toxic ion resistance (LMO1967). During the same period, the induced stress proteins were DNA binding protein (fri), organic hydroperoxide resistance protein (LMO2199), arsenate reductase (LMO2230), and a universal stress protein (LMO1580). After 120 min, an additional six stress related genes were repressed. These included hrcA (a negative regulator of class I heat shock genes), a general stress protein (LMO1601), a protein related to oxidative stress (msrB), and three genes involved in DNA recombination and repair, single strand binding protein (ssb), an endonuclease involved in recombination (LMO1502), and exonuclease ABC subunit A (urvA). Three ATP-dependent endopeptidases needed for protein turnover (clpE, clpB, clpX) were also repressed. At T₃₀ one transcription regulator of the marR family (LMO2200), which is a negative regulator of antibiotic resistance proteins in E. coli was induced, while at T₁₂₀ another transcription of the same family (LMO0266) was repressed.

SP 25A repressed key genes involved in cell division. After 120 min, four proteins involved in cell division were repressed; two DNA gyrase subunits, one ATPase associated with chromosome replication, and one gene that encoded a cell division initiation protein, DivIVA, which is crucial for the initiation of cell division. Repression of DivIVA alone would be enough to inhibit cell division. Exposure to RLs resulted in only a small reduction in cell density (FIGS. 6A-6B). In contrast to SP 25A, exposure to RLs did not affect the expression of any of the genes encoding for proteins involved in cell division.

Exposure of L. monocytogenes to SP 25A led to repression of lytR. Repression of lytR is related to a decrease in autolytic enzyme activity. The exposure of S. mutans to SP 25A resulted in inhibition of cell division. The information gathered from the gene profiling experiment uncovered a regulatory link between lytR and DivIVA (FIG. 6A, Table 6). These observations indicate that the inhibition of growth, rather than membrane disruption, is the mode of action for SP 25A.

Further analysis of the gene profiling experiments reveal that exposure to SP 25A causes significant changes in intermediary metabolism, especially glycolysis and pathways needed for energy production (Table 6). Repression of central metabolism would lead to a lack of enzymes needed for generation of precursor metabolites and energy needed for growth. Repression of pyruvate dehydrogenase complex (8- to 16-fold), pyruvate kinase (1.6 fold), and phosphoglyceromutase (1.5 fold) would virtually stop energy production from glycolysis.

Glycolytic intermediates are also important for generation of acetyl CoA, pyruvate and phosphoglycerate, all being precursor metabolites for production fatty acids and amino acids. Exposure to RLs induced expression of the E3 subunit of pyruvate dehydrogenase, phosphoglyceromutase, and two more enzymes in the pentose phosphate pathway, indicating that glycolysis was induced and energy production improved. This may explain the reduction in membrane permeabilization after 120 min of exposure to RLs. In contrast to the effects of exposure to RLs, addition of SP 25A to the growth media caused a 1.5- to 3-fold repression of five enzymes in the pentose phosphate pathway. The lack of induction of alternative pathways for formation of the metabolites generated from the pentose phosphate pathway left the cell with no method to produce energy or intermediates to use in cell division. The repression of several key enzymes of central intermediary metabolism was also observed in E. coli when challenged with sub-MIC doses of Bac7. Amongst the repressed genes were genes encoding for sugar transporters and glycolytic intermediates, leading to inhibition of cell growth.

SP 25A led to approximately a 5-fold repression of four proteins needed for the synthesis of Fe-S clusters. Fe-S clusters are essential in multiple diverse reactions, including electron transport, regulation of gene expression, and mediation of redox as well as non-redox catalysis. SP 25A also caused down regulation of two subunits of the proton pump by 1.5-fold and quinol oxidase by 2-fold, leading to disruption of the oxidative phosphorylation machinery. RLs, in contrast, led to a 3-fold induction of one subunit of proton pump (atpE) at T₃₀ and a 3.5-fold repression at T₁₂₀. Hence, after 120 min SP 25A repressed the cells ability to generate precursor metabolites, as well as energy, while RLs did not have that effect at all. In E. coli, repression of iron metabolism was found only in transport (fecA), other genes were not affected.

Genes associated with transcription and translation were repressed between 1.5- and 12-fold resulting from the addition of SP 25A to the growth media. SP 25A led to the repression of three out of four RNA polymerase subunits by 1.5- to 2-fold, which completely disrupted transcription. As a result of this decrease in transcription, translation activity was also repressed. The effect upon genes related to translation was widespread with repression (1.5- to 4-fold) of 11 ribosomal genes after 120 min, and two elongation factors (2.5- to 12-fold). Contrary to this study, Tomasinsig et al. (Tomasinsig L., Scocchi M., Mettulio R., and Zanetti M., 2004, Antimicrob. Agents Chemother. 48:3260-3267) observed an induction of ribosomal genes after exposure to a proline rich antibacterial peptide.

Widespread regulation of stress-related genes occurred upon addition of SP 25A to the growth media (see Table 6). Multiple systems were regulated during the time that the microbes were exposed to SP 25A. These regulated systems include genes related to osmotic regulation, DNA repair, chaperones, and peroxide resistance. For example, the large conductance mechanosensitive channel (mscL) was induced at T₃₀, but was repressed at T₁₂₀. This gene is associated with hypo-osmotic shock, likely caused by the interaction of SP 25A with the membrane. Induction of mscL demonstrates the cells effort to modulate the osmotic change due to the membrane disruption caused by the addition of SP 25A. Repression of the membrane protein at T₁₂₀ likely indicates that the cell is no longer under osmotic stress. This explanation seems likely, considering the membrane permeabilization decline at T₁₂₀. No other group has observed this phenomenon in response to an antimicrobial peptide, despite observing changes in membrane and transport proteins associated with sugar and ion flux.

Interestingly, in all cases, stress-associated genes were repressed after 120 min of exposure to SP 25A. This may indicate that the cell has adapted to the effects of SP 25A, but it may also represent the inability to produce new RNA and proteins with the repression of the transcription and translation apparatus observed in this study. Three genes encoding for Clp ATPases were repressed upon exposure to SP 25A. Clp ATPases are implicated in regulation of cellular stress responses due to their protein reactivation, remodeling activities, and their capacity to target misfolded proteins. There are no reports in literature of stress related genes being repressed in response to exposure with antimicrobial peptides.

Four virulence genes essential for intracellular survival of L. monocytogenes were repressed with treatment of SP 25A after 120 min of exposure. The gene encoding for fibronectin binding protein and hly were induced while iap was repressed at T₃₀, but after 120 min each of these genes in addition to plcA were repressed. In contrast, addition of RLs induced hlyA expression after 120 min. Down-regulation of these genes would make L. monocytogenes EGDe less virulent by repressing translation and expression of the binding proteins and impairing the capacity for intracellular survival.

The effective reduction in cell density after treatment with SP 25A was associated with the repression of key genes involved in cell division and genome replication. Therefore, one possible mode of action for SP 25A is through inhibiting cell division. SP 25A also repressed key genes in central metabolism, generation of precursor metabolites, transcription, and translation, resulting in repression of RNA production, protein biosynthesis, cellular energy, and virulence. In contrast, RLs only affected a few genes in any given functional category that were not associated with any single phenotypic observation. Therefore, SP 25A caused repression in the cell's metabolism, which was independent of the observed pore forming activity. Taken together, these data led us to conclude that RLs and SP 25A, even though acting on the cell membrane, produced distinctly different gene expression profiles with SP 25A being more effective for inhibiting cell growth in L. monocytogenes.

### Example 9

### Anticancer Assays

Cancer cell assays can be performed using the MTT dye protocol. Viability of cells may be determined by the dye response. In the following procedure, approximately 1.5×10⁴ cells per well can be added and viability may be determined with the cells in a semi-confluent state. The assay can be performed in a 96-well microtiter plate using a New Brunswick Cell Counter. After addition of the compound or compounds of the present invention, the plate may incubate for 1-24 hours. MTT may be added to each well including negative control wells untreated with the compounds of the present invention. The plate would then be incubated at 37° C. for 4 hours. The liquid contents of each well can then be removed, and isopropanol with 0.1 M HCl would then be added to each well. The plate can be sealed with parafilm to prevent evaporation of the isopropanol. The plate would then be allowed to sit for approximately 5-10 minutes in order to solubilize the precipitate. Purified water would then be added to each well. Results for each concentration of compound or compounds can be plotted relative to untreated controls, and IC₅₀ values can be therefrom determined (Table 9).

### Example 10

### Stimulation and Proliferation of Leukocytes

In vitro viability of human leukocyte cells in the presence of different compounds of the present invention, including but not limited to pharmaceutical compositions, at different concentrations can be determined by an Alamar Blue protocol. Alamar Blue is an indicator dye, formulated to measure quantitatively the proliferation and cytotoxicity of the cells. The dye consists of an oxidation-reduction (redox) indicator that yields a calorimetric change and a fluorescent signal in response to cellular metabolic activity.

Blood from a patient may be drawn and centrifuged at room temperature. The buffy coat cells at the plasma-red blood cell interface may then be aspirated. Buffy coat cells (mainly lymphocyte cells) can then be transferred into centrifuge tubes containing Fetal Bovine Serum. The buffy coat suspension may then be carefully layered on Histopaque and centrifuged at room temperature. The interface, which is mostly PBMCs (peripheral mononuclear cells), can then be aspirated and transferred into a appropriate tube, and resuspended. The resulting slurry can then be centrifuged. The supernatant may then be aspirated and discarded. The cell pellet can then be re-suspended. The cell counts can then be performed with a hemocytometer.

The Alamar Blue stain used in the aforementioned example permeates both cell and nuclear membranes, and is metabolized in the mitochondria to cause the change in color. The resulting fluorometric response can be a result of total mitochondrial activity caused by cell stimulation and/or mitosis (cell proliferation). The increase in values (for treated cells, as a percent of values for untreated cells) with increased incubation time may be attributed to increased cell proliferation in addition to stimulation of cell metabolic activity caused by the compound or compounds of the present invention.

The compounds of the present invention which cause stimulation and proliferation of leukocytes may be active upon both the phagocytic and lyphocyte cell components of the mammalian lymphatic system. As such, certain of the compositions of the compounds of the present invention which are relatively non-toxic to mammalian cells at therapeutic dose levels may be used as immunomodulators to treat humans or other mammals with compromised immune systems. Such treatment may be administered systemically in vivo or by extra-corporeal treatment of whole blood or blood components to be reinfused to the donor. Such therapy may serve to counteract immune deficiency in neutropenic patients caused by age, disease, or chemotherapy and could stimulate natural immune responses to prevent or combat pathogenic infections and growth of certain cancer cell lines or to enhance wound healing processes involving the lymphoid system.

The compounds of the present invention may also be useful in the concomitant treatment of bacterial infections associated with viral infections such as AIDS.

### Example 11

### Tablet Formulation

The following ingredients are mixed intimately and pressed into single scored tablets: 400 mg compound(s) of the present invention, 50 mg cornstarch, 25 mg croscarmellos, 25 mg sodium lactose, 120 mg magnesium stearate.

### Example 12

### Capsule Formulation

The following ingredients can be mixed intimately and loaded into a hard-shell gelatin capsule: 200 mg compound(s) of the present invention, 200 mg lactose, 150 mg spray-dried magnesium stearate.

### Example 13

### Suspension Formulation

The following ingredients can be mixed to form a suspension for oral administration: 1.0 g compound(s) of the present invention, 1.0 g furmaric acid, 0.5 g sodium chloride, 2.0 g methyl paraben, 0.15 g propyl paraben, 0.05 g granulated sugar, 25.0 g sorbitol (70% solution), 13.00 g Veegum K (Vanderbilt Co.), 1.0 g flavoring, 0.035 ml colorings, distilled water to bring the total volume to 100 mL.

### Example 14

### Injectable Formulation

The following ingredients can be mixed to form an injectable formulation: 10 mg compound(s) of the present invention, 0.2 mg-20 mg sodium acetate buffer solution, 0.4 M 2.0 ml HCl (1 N) or NaOH (1 N) adjusted to a suitable pH, and water (distilled, sterile) to bring the total volume to 20 mL.

### Example 15

### Suppository Formulation

A suppository of total weight 2.5 g may be prepared by mixing 190 mg of compound(s) of the invention with 2.31 g of Witepsol™ H-15 (triglycerides of saturated vegetable fatty acid; Riches-Nelson, Inc., New York).

### Example 16

### Therapeutic Composition

A therapeutic composition can be formulated in accordance with the present invention. The composition can be prepared by mixing 7.5% syringopeptin 25A, 7.5% rhamnolipids, and 85% saline solution, where all percentages represent the amount per weight of the final solution. The saline solution can be formulated to include 0.5% sodium chloride. The solution can be mixed so that the components become homogeneously distributed with one another, which can be denoted as Formulation 1A (see Table 7, which shows therapeutic solutions of RLs and SP 25A). The composition may then be loaded into a syringe so that it can be administered by injection.

### Example 17

### Therapeutic Solutions

RLs and SP 25A solutions containing various components and associated concentrations can be prepared in accordance with the procedure described in example 16 (see Table 7).

### Example 18

### Therapeutic Gels

A therapeutic topical gel can be formulated in accordance with the present invention. The gel can be prepared by mixing 7.5% syringopeptin 25A, 7.5% rhamnolipid, 30% cellulose gum, 10% calcium glycerol phosphate, and 44.8% purified water, where all percentages represent the amount per weight of the final gel. Additionally, grapefruit seed extract may be added as a preservative at 0.2% of the gel. The mixture can then be mixed so that the components become homogeneously distributed with one another to form a gel, which can be denoted as Formulation 1B. The gel can then be loaded into a squeeze-container so that it can be administered drop-wise to a wound having a microbial infection.

Therapeutic compositions containing various components and associated concentrations are prepared in accordance with the procedure described above. The resulting compositions are illustrated in Table 8.

### Example 19

### Agar Proportion Method

The Agar Proportion Method for determining drug susceptibility to M. tuberculosis can be prepared by the following procedure. Briefly, Middlebrook 7H10 medium with OADC supplement (DIFCO) is incorporated with the RL and/or SP at various concentrations. Fresh growth of the test organism on Lowenstein Jensen medium is used as the source of the inoculum. Sufficient number of colonies are picked up to make a suspension equivalent to McFarland standard 1. Quadrant plates can be used for the Agar Proportion Method and 0.01 ml of each dilution (10⁻¹ and 10⁻²) of the inoculum is placed in each quadrant. A control plate is also inoculated with the undiluted suspension. The plates are inoculated at 37° C. in the presence of 5-10% CO₂ for about 3 weeks. The drug susceptibility test results can obtained after 3 weeks by comparing the number of colony forming units growing on the medium containing the RL and/or SP by being compared to the control plate. The proportion of resistant cells in the total viable population of the original inoculum is then calculated and expressed as a percentage.

### Example 20

### Etest

The Etest for determining drug susceptibility to M. tuberculosis can be prepared by the following procedure. Briefly, Etest strips containing gradients of Isoniazid (e.g., 0.016-256 ug/ml) and Rifampicin (e.g., 0.016-256 ug/ml) can be purchased from AB BIODISK, Sweden. Similar RL strips and/or SP strips can be prepared by using paper strips or litmus strips soaked in different compositions comprising gradients of RL and SP to form a series of RL strips and a series of SP strips. Also, a series of RL-SP strips having different concentrations of RL and/or SP can be made. It is advantageous if the RL strips and/or SP strips are labeled with the concentration impregnated therein. The inoculums can be prepared substantially the same as described in Example 19. The inoculum is prepared and swabbed directly onto 7H10 Agar plates. The plates are then pre-incubated for 18 hours at 37° C. prior to placing Etest strips RL strips, SP strips, and/or RL-SP strips on the plates followed by further incubation until growth is visible (in approximately 7-10 days) in at least the control. The inhibition of colony formation or growth is an indication that the RL and/or SP at the concentration of the strip can be used in inhibit M. tuberculosis. The MIC is interpreted as the point at which the ellipse intersects the Etest strip. The cut-off value above which the isolate is labeled resistant is about 0.2 ug/ml for Isoniazid and 1 mg/ml for Rafampicin. The RL strips, SP strips, and/or RL-SP strips can then be compared to the Etest strip, and the RL strip, SP strip, and/or RL-SP strip having the lowest concentration that compares with Etest strip can provide an indication of the MIC.

TABLE 1
Table 1. List of bacteria used for antimicrobial screening and their growth conditions.

| | | Temperature | Oxygen | |
|---|---|---|---|---|
| Organism | Strain | (° C.) | Demand | Medium |
| Aeromonas caviae | 13137 | 30 | Aerobic | Nutrient |
| | | | | agar |
| Bacillus cereus | 10987 | 30 | Aerobic | Nutrient |
| | | | | agar |
| Bacillus subtilis | 23857 | 26 | Aerobic | Nutrient |
| | | | | agar |
| Bacillus megaterium | 14581 | 30 | Aerobic | NB |
| Brevibacterium linens | BL1MGE | 37 | Aerobic | TSB |
| Citrobacter fruendii | 11811 | 37 | Aerobic | Nutrient |
| | | | | agar |
| Clostridium sporogenes | 10000 | 37 | Anaerobic | Reinforced |
| | | | | clostridial |
| | | | | medium |
| Enterobacter aerogenes | 13048 | 30 | Aerobic | Nutrient |
| | | | | agar |
| Enterococcus faecalis | 700802 | 37 | Aerobic | BHI |
| Erwinia herbicola | 33243 | 37 | Aerobic | Nutrient |
| | | | | agar |
| Eschereschia coli | K12 | 37 | Aerobic | Nutrient |
| | | | | agar |
| Eschereschia coli H7:0157 | 35150 | 37 | Aerobic | Nutrient |
| | | | | agar |
| Flavobacterium devorans | 10829 | 30 | Aerobic | Nutrient |
| | | | | agar |
| Klebsiella pneumoniae sub sp | 700721 | 37 | Aerobic | NB |
| pneumoniae | | | | |
| Lactobacillus plantarum | 8014 | 37 | Microaerophilic | MRS |
| Lactobacillus acidophilus | 4355 | 37 | Microaerophilic | MRS |
| Lactococcus lactis subsp lactis | IL1403 | 30 | Microaerophilic | Ellikers |
| | | | | Broth |
| Listeria innocua | 33090 | 37 | Aerobic | BHI |
| Listeria monocytogenes | 43251 | 37 | Aerobic | BHI |
| Micrococcus luteus | 21102 | 30 | Aerobic | BHI |
| Mycobacterium smegmatis | 14468 | 37 | Aerobic | Luria |
| | | | | Broth |
| Salmonella typhimurium | 13076 | 37 | Aerobic | Nutrient |
| | | | | agar |
| Salmonella enteridis | 700931 | 37 | Aerobic | TSB |
| Staphylococcus aureus subsp | 700699 | 37 | Aerobic | BHI |
| aures | | | | |
| Streptococcus mutans | 89/1591 | 37 | Aerobic | BHI |
| Streptococcus suis | 700610 | 37 | Aerobic | BHI |
| Streptococcus agalacticae | 12403 | 37 | Aerobic | BHI |
| Bacillus subtilis (spores) | 6633 | 26 | Aerobic | TSB |
| Clostridium | 11437 | 37 | Anaerobic | Reinforced |
| sporogenes (spores) | | | | clostridial |
| | | | | medium |

TABLE 2
Table 2. MICs and mean IR's of SP 25A and rhamnolipids
against screened organisms.

| | IR | | | |
|---|---|---|---|---|
| | Rhamnolipids | MIC | IR SP 25A | MIC |
| Genus | (60 μg/mL) | (μg/mL) | (50 μg/mL) | (μg/mL) |
| Bacillus megaterium | 1.043 | 4 | 0.005 | 3 |
| Listeria innocua | 0.014 | 5 | 0.005 | 3 |
| Listeria monocytogenes | 0.032 | 6 | 0.005 | 3 |
| Bacillus cereus | 0.834 | 4 | 0.004 | 4 |
| Bacillus subtilis | 1.807 | 4 | 0.006 | 4 |
| Clostridium sporogenes | 0.698 | 4 | 0.008 | 4 |
| Flavobacterium devorans | 0.518 | 16 | 0.002 | 4 |
| Lactococcus lactis subsp. Lactis | 1.219 | 4 | 0.008 | 4 |
| Micrococcus luteus | 0.183 | 8 | 0.006 | 4 |
| Mycobacterium smegmatis | ND | 4 | ND | 4 |
| Streptococcus mutans | 0.164 | 4 | 0.003 | 4 |
| Streptococcus suis | 1.018 | 4 | 0.006 | 4 |
| Bacillus subtilis (spores) | ND | 4 | ND | 4 |
| Clostridium sporogenes (spores) | ND | 4 | ND | 4 |
| Enterococcus faecalis | 0.482 | >60 | 0.001 | 8 |
| Lactobacillus acidophilus | 0.196 | 16 | 0.003 | 8 |
| Staphylococcus aureus subsp. aureus | 0.894 | >60 | 0.003 | 8 |
| Streptococcus agalacticae | 1.073 | 4 | 0.004 | 8 |
| Lactobacillus plantarum | 0.287 | 32 | 0.003 | 16 |
| Aeromonas caviae | 0.000 | >60 | 0.000 | >50 |
| Citrobacter fruendii | 0.000 | >60 | 0.000 | >50 |
| Enterobacter aerogenes | 0.000 | >60 | 0.000 | >50 |
| Erwinia herbicola | 0.000 | >60 | 0.000 | >50 |
| Eschereschia coli K12 | 0.000 | >60 | 0.000 | >50 |
| Klebsiella pneumoniae subsp. | 0.000 | >60 | 0.000 | >50 |
| Pneumoniae | | | | |
| Salmonella typhimurium | 0.000 | >60 | 0.000 | >50 |
| Salmonella enteridis | 0.000 | >60 | 0.000 | >50 |
| Brevibacterium linens | 0.512 | ND | 0.009 | ND |
| Eschereschia coli H7:0157 | 0.000 | ND | 0.000 | ND |

(ND = Not Determined).

TABLE 3
Table 3. Functional categories that contained the genes that were significantly
differentially expressed in response to treatment with sub-MIC doses of RLs.

| | Total | No. of differentially expressed genes | | | |
|---|---|---|---|---|---|
| | genes in | T₃₀ | | T₁₂₀ | |
| Functional category | category | Induced | Repressed | Induced | Repressed |
| ABC transporters | 134 | 0 | 0 | 2 | 1 |
| Ion channels | 4 | 0 | 0 | 0 | 0 |
| PEP/PTS components | 80 | 0 | 0 | 3 | 0 |
| Polysaccharide degradation | 18 | 0 | 0 | 1 | 0 |
| Central Intermediary metabolism | 272 | 0 | 1 | 4 | 0 |
| Cofactor and coenzyme metabolism | 116 | 0 | 0 | 0 | 0 |
| Amino acid metabolism | 179 | 0 | 0 | 2 | 0 |
| Electron transport and oxidative | 61 | 1 | 0 | 0 | 1 |
| phosphorylation | | | | | |
| Cell wall metabolism | 57 | 0 | 0 | 0 | 0 |
| Transcription regulators | 142 | 1 | 0 | 2 | 0 |
| Transcription | 36 | 0 | 0 | 0 | 0 |
| Protein biosynthesis | 158 | 1 | 0 | 1 | 1 |
| Protein fate | 92 | 0 | 1 | 1 | 0 |
| Secretion | 63 | 0 | 0 | 1 | 0 |
| Virulence factors | 131 | 0 | 0 | 1 | 0 |
| Stress | 50 | 0 | 0 | 2 | 0 |
| Cell division | 17 | 0 | 0 | 0 | 0 |
| Phage proteins | 18 | 1 | 0 | 0 | 2 |
| Unknown/Hypothetical proteins | — | 4 | 0 | 7 | 2 |
| Total | 1628 | 8 | 2 | 27 | 7 |

TABLE 4
Table 4. Significantly differentially regulated genes during exposure to RLs.

| | | | Log₂ Ratios | | |
|---|---|---|---|---|---|
| Protein | ORF | Cellular Role | T₃₀ | T₁₂₀ | Q value |
| ABC transporter (Metal binding protein) | LMO1073 | ABC transporters | −0.18 | 0.97 | 0.26 |
| ABC transporter (ATP-binding protein) | LMO2193 | ABC transporters | 0.33 | 0.32 | 0.11 |
| ABC transporter-associated protein, sufB | LMO2411 | ABC transporters | −0.43 | −0.17 | 0.23 |
| Fructose-specific phosphotransferase | LMO0399 | PEP/PTS components | 0.21 | 0.38 | 0.11 |
| enzyme IIB | | | | | |
| Fructose-specific phosphotransferase | LMO0400 | PEP/PTS components | 0.05 | 2.54 | 0.11 |
| enzyme IIC | | | | | |
| Beta-glucoside-specific enzyme IIABC | LMO0738 | PEP/PTS components | 0.46 | 1.36 | 0.11 |
| component | | | | | |
| Alpha mannosidase | LMO0401 | Polysaccharide | −0.15 | 2.70 | 0.11 |
| | | degradation | | | |
| Ribose 5-phosphate isomerase | LMO0736 | Intermediary | −0.03 | 1.72 | 0.11 |
| | | metabolism | | | |
| 6-phospho-beta-glucosidase | LMO0739 | Intermediary | 0.04 | 2.86 | 0.11 |
| | | metabolism | | | |
| Pyruvate dehydrogensae (dihydrolipoamide | LMO1055 | Intermediary | 0.24 | 0.96 | 0.11 |
| dehydrogenase, E3 subunit), pdhD | | metabolism | | | |
| Phosphoglyceromutase | LMO2205 | Intermediary | −0.73 | 0.86 | 0.15 |
| | | metabolism | | | |
| Ribulose-phosphate 3-epimerase | LMO2659 | Intermediary | 0.01 | 1.70 | 0.11 |
| | | metabolism | | | |
| Glycine dehydrogenase (decarboxylating) | LMO1350 | Amino acid | −0.41 | 1.21 | 0.11 |
| subunit 2 | | metabolism | | | |
| Threonine 3-dehydrogenase | LMO2663 | Amino acid | −0.06 | 1.98 | 0.11 |
| | | metabolism | | | |
| H+-transporting ATP synthase C chain, | LMO2534 | Respiration and | 1.64 | −1.81 | 0.11 |
| atpE | | oxidative | | | |
| | | phosphorylation | | | |
| Peroxide operon regulator, perR | LMO1683 | Transcription regulator | −0.13 | 1.03 | 0.11 |
| Transcriptional regulatory protein degU | LMO2515 | Transcription regulator | 0.12 | 1.39 | 0.16 |
| Phenylalanyl-tRNA synthetase alpha chain, | LMO1221 | Protein biosynthesis | 0.62 | −0.45 | 0.29 |
| pheS | | | | | |
| Acetyltransferase | LMO0624 | Post translational | −0.62 | 1.01 | 0.29 |
| | | modification | | | |
| ATP-dependent endopeptidase clp ATP- | LMO0997 | Protein degradation | −0.03 | 1.28 | 0.11 |
| binding subunit, clpE | | | | | |
| Oxidoreducatse involved in TATpathway | LMO0737 | Secretion | 0.05 | 1.02 | 0.11 |
| secreted proteins | | | | | |
| listeriolysin O precursor, hly | LMO0202 | Virulence | 0.24 | 1.90 | 0.11 |
| Single-stranded DNA-binding protein, ssb | LMO0045 | Stress | −0.01 | 1.33 | 0.11 |
| Cold shock protein, cspL | LMO1364 | Stress | 0.14 | 0.45 | 0.29 |
| Non-heme iron-binding ferritin, fri | LMO0943 | Stress | 0.09 | 0.81 | 0.11 |
| Phage proteins | LMO2287 | Phage proteins | 0.66 | −0.58 | 0.11 |
| Phage proteins | LMO2327 | Phage proteins | 0.27 | −0.61 | 0.11 |
| Hypothetical Protein | LMO0743 | Unknown | 0.12 | 0.82 | 0.15 |
| Hypothetical Protein | LMO1113 | Unknown | 0.77 | −0.39 | 0.11 |
| Hypothetical Protein | LMO2257 | Unknown | 0.00 | 0.94 | 0.11 |
| Hypothetical Protein | LMO2432 | Unknown | −0.20 | 3.24 | 0.11 |
| Stage V sporulation protein G | LMO0197 | Others | 0.86 | −0.61 | 0.11 |
| Rhodanese-related sulfurtransferases | LMO1384 | Others | 0.60 | −0.48 | 0.11 |
| Glycerol uptake facilitator protein | LMO1539 | Others | 0.21 | 0.82 | 0.14 |
| Creatinine amidohydrolase family protein | LMO1968 | Others | 0.77 | −0.56 | 0.11 |
| Protease I | LMO2256 | Others | 0.17 | 0.99 | 0.26 |
| Putative transcriptional regulator, MerR | LMO2728 | Others | −0.05 | 0.63 | 0.15 |
| family | | | | | |
| Putative transcriptional regulator, MerR | LMO2334 | Others | 0.57 | −0.66 | 0.15 |
| family | | | | | |

TABLE 5
Table 5. Functional categories that contained the genes that were significantly
differentially expressed in response to treatment with sub-MIC doses of SP 25A.

| | Total | Number of differentially expressed genes | | | |
|---|---|---|---|---|---|
| | genes in | T₃₀ | | T₁₂₀ | |
| Functional category | category | Induced | Repressed | Induced | Repressed |
| ABC transporters | 134 | 1 | 3 | 2 | 8 |
| Ion channels | 4 | 1 | 0 | 0 | 1 |
| PEP/PTS components | 80 | 0 | 4 | 0 | 4 |
| Polysaccharide degradation | 18 | 0 | 1 | 0 | 1 |
| Central Intermediary metabolism | 272 | 2 | 13 | 0 | 24 |
| Cofactor and coenzyme metabolism | 116 | 0 | 4 | 0 | 5 |
| Amino acid metabolism | 179 | 0 | 1 | 0 | 4 |
| Electron transport and oxidative | 61 | 0 | 4 | 0 | 5 |
| phosphorylation | | | | | |
| Cell wall metabolism | 57 | 0 | 1 | 0 | 2 |
| Transcription regulators | 142 | 1 | 0 | 1 | 4 |
| Transcription | 36 | 0 | 0 | 0 | 3 |
| Protein biosynthesis | 158 | 2 | 3 | 0 | 12 |
| Protein fate | 92 | 0 | 3 | 0 | 4 |
| Secretion | 63 | 1 | 2 | 0 | 3 |
| Virulence factors | 131 | 2 | 1 | 0 | 4 |
| Stress | 50 | 4 | 5 | 0 | 13 |
| Cell division | 17 | 0 | 2 | 0 | 4 |
| Phage proteins | 18 | 0 | 0 | 0 | 0 |
| Unknown/Hypothetical proteins | — | 12 | 13 | 2 | 34 |
| Total | 1628 | 26 | 60 | 5 | 135 |

TABLE 6
Table 6. Significantly differentially regulated genes during exposure to SP 25A.

| | | | Log₂ Ratios | | |
|---|---|---|---|---|---|
| Protein | ORF | Cellular Role | T₃₀ | T₁₂₀ | Q value |
| Manganese uptake Mn ABC transporter | LMO1847 | ABC transporters | 0.66 | −3.85 | 0.00 |
| Metal cations ABC transporter, permease | LMO1848 | ABC transporters | −3.43 | −2.43 | 0.00 |
| protein | | | | | |
| ABC transporter, ATP-binding protein | LMO2415 | ABC transporters | −0.47 | −0.65 | 0.00 |
| Heavy metal-transporting ATPase | LMO0641 | ABC transporters | −0.45 | −0.42 | 0.01 |
| Manganese transport proteins NRAMP | LMO1424 | ABC transporters | −2.10 | 0.01 | 0.01 |
| Metal cations ABC transporter, ATP- | LMO1849 | ABC transporters | −0.89 | −0.78 | 0.04 |
| binding proteins | | | | | |
| ABC transporter-associated protein | LMO2411 | ABC transporters | −0.51 | −0.20 | 0.04 |
| (sufB) | | | | | |
| Oligopeptide ABC transporter (ATP- | LMO2193 | ABC transporters | −0.24 | −0.46 | 0.17 |
| binding protein) | | | | | |
| Acetoin uptake permease protein | LMO2239 | ABC transporters | −0.24 | 0.68 | 0.17 |
| ABC transporter (ATP-binding protein) | LMO2139 | ABC transporters | 0.07 | 0.60 | 0.23 |
| Large conductance mechanosensitive | LMO2064 | Ion Channel | 1.96 | −2.70 | 0.00 |
| channel | | | | | |
| Fructose-specific phosphotransferase | LMO0400 | PEP/PTS components | −1.78 | −0.34 | 0.00 |
| enzyme IIC | | | | | |
| Beta-glucoside-specific enzyme IIABC | LMO2373 | PEP/PTS components | 0.39 | −0.83 | 0.00 |
| component | | | | | |
| Beta-glucoside-specific enzyme IIABC | LMO0738 | PEP/PTS components | −1.04 | −1.04 | 0.00 |
| component | | | | | |
| Trehalose specific enzyme IIBC | LMO1255 | PEP/PTS components | −3.74 | −0.40 | 0.00 |
| Alpha mannosidase | LMO0401 | Polysaccharide | −2.61 | −0.78 | 0.00 |
| | | degradation | | | |
| 6-phospho-beta-glucosidase | LMO0739 | Intermediary metabolism | 0.97 | −2.39 | 0.00 |
| 6-phospho-beta-glucosidase | LMO0536 | Intermediary metabolism | −0.38 | −0.24 | 0.23 |
| Alpha,alpha-phosphotrehalase | LMO1254 | Intermediary metabolism | −2.10 | −0.04 | 0.00 |
| Pyruvate dehydrogenase (E1 alpha | LMO1052 | Intermediary metabolism | −2.74 | −0.38 | 0.00 |
| subunit), pdhA | | | | | |
| Pyruvate dehydrogenase (E1 beta | LMO1053 | Intermediary metabolism | −3.04 | −1.38 | 0.00 |
| subunit), pdhB | | | | | |
| Pyruvate dehydrogenase | LMO1054 | Intermediary metabolism | −2.04 | −2.62 | 0.00 |
| (dihydrolipoamide acetyltransferase E2 | | | | | |
| subunit), pdhC | | | | | |
| Pyruvate dehydrogensae | LMO1055 | Intermediary metabolism | −2.58 | −2.31 | 0.00 |
| (dihydrolipoamide dehydrogenase, E3 | | | | | |
| subunit), pdhD | | | | | |
| L-lactate dehydrogenase, ldh | LMO1057 | Intermediary metabolism | −1.11 | −0.52 | 0.04 |
| Pyruvate kinases, pykA | LMO1570 | Intermediary metabolism | −0.60 | 0.38 | 0.03 |
| Ribose 5-phosphate isomerase | LMO0736 | Intermediary metabolism | −0.81 | −0.32 | 0.00 |
| Ribulose-5-Phosphate 3-Epimerase | LMO0735 | Intermediary metabolism | −0.43 | −0.48 | 0.00 |
| Transaldolase | LMO2743 | Intermediary metabolism | −0.62 | −0.13 | 0.29 |
| Fructose-1,6-bisphosphate aldolase, fbaA | LMO2556 | Intermediary metabolism | −0.22 | −0.38 | 0.07 |
| Dihydroxyacetone kinase | LMO2695 | Intermediary metabolism | −0.49 | −0.71 | 0.03 |
| Dihydroxyacetone kinase | LMO2696 | Intermediary metabolism | −0.51 | −0.81 | 0.00 |
| Dihydroxyacetone kinase | LMO2697 | Intermediary metabolism | 0.41 | −1.51 | 0.01 |
| phosphotransfer protein | | | | | |
| Glucosamine-6-Phoasphate isomerase | LMO0957 | Intermediary metabolism | −0.52 | −0.31 | 0.07 |
| L-glutamine-D-fructose-6-phosphate | LMO0727 | Intermediary metabolism | 0.76 | −2.90 | 0.00 |
| amidotransferase | | | | | |
| Phosphoglyceromutase | LMO2205 | Intermediary metabolism | −0.21 | −0.42 | 0.03 |
| Branched-chain alpha-keto acid | LMO1374 | Amino acid metabolism | −0.56 | −0.11 | 0.03 |
| dehydrogenase E2 subunit (lipoamide | | | | | |
| acyltransferase) | | | | | |
| Glycerate dehydrogenases | LMO1684 | Amino acid metabolism | −1.26 | −1.22 | 0.00 |
| Glycine dehydrogenase (decarboxylating) | LMO1350 | Amino acid metabolism | −0.34 | −1.68 | 0.00 |
| subunit 2 | | | | | |
| Alanine dehydrogenase | LMO1579 | Amino acid metabolism | −0.38 | −0.25 | 0.00 |
| IscU protein | LMO2412 | Cofactor-coenzyme | −1.39 | −1.00 | 0.00 |
| | | metabolism | | | |
| Cysteine desulfurase | LMO2413 | Cofactor-coenzyme | −2.04 | −0.40 | 0.10 |
| | | metabolism | | | |
| SufD protein | LMO2414 | Cofactor-coenzyme | −1.50 | −0.94 | 0.00 |
| | | metabolism | | | |
| Pyridoxine biosynthesis protein | LMO2101 | Cofactor-coenzyme | −1.82 | −0.40 | 0.00 |
| | | metabolism | | | |
| Pyridoxine biosynthesis amidotransferase | LMO2102 | Cofactor-coenzyme | −0.15 | −0.83 | 0.00 |
| | | metabolism | | | |
| AA3-600 quinol oxidase subunit I | LMO0014 | Respiration and | −0.91 | −0.09 | 0.10 |
| | | oxidative | | | |
| | | phosphorylation | | | |
| AA3-600 quinol oxidase subunit III | LMO0015 | Respiration and | −0.73 | −0.50 | 0.03 |
| | | oxidative | | | |
| | | phosphorylation | | | |
| AA3-600 quinol oxidase subunit IV | LMO0016 | Respiration and | −0.52 | −0.37 | 0.17 |
| | | oxidative | | | |
| | | phosphorylation | | | |
| H+-transporting ATP synthase chain | LMO2531 | Respiration and | −0.58 | 0.11 | 0.29 |
| alpha, atpA | | oxidative | | | |
| | | phosphorylation | | | |
| H+-transporting ATP synthase C chain, | LMO2534 | Respiration and | −1.14 | 0.32 | 0.00 |
| atpE | | oxidative | | | |
| | | phosphorylation | | | |
| UDP-N-acetylglucosamine 1- | LMO2526 | Cell wall metabolism | −1.10 | −0.21 | 0.00 |
| carboxyvinyltransferase, murA | | | | | |
| Peptidoglycan anchored protein (LPXTG | LMO2714 | Cell wall metabolism | −0.03 | −0.98 | 0.00 |
| motif) | | | | | |
| Transcriptional regulator, MarR family | LMO0266 | Transcription regulator | −0.25 | 0.89 | 0.29 |
| Transcriptional regulator, MarR family | LMO2200 | Transcription regulator | 0.62 | −2.07 | 0.00 |
| Transcriptional regulator, LytR family | LMO0433 | Transcription regulator | −0.53 | −0.80 | 0.01 |
| Heat-inducible transcription repressor, | LMO1475 | Transcription regulator | −0.56 | −0.16 | 0.00 |
| hrcA | | | | | |
| Peroxide operon regulator, perR | LMO1683 | Transcription regulator | 0.28 | −2.71 | 0.00 |
| Negative regulator of genetic competence | LMO2190 | Transcription regulator | 0.24 | −0.62 | 0.02 |
| mecA | | | | | |
| RNA polymerase (alpha subunit), rpoA | LMO2606 | Transcription | −0.31 | −0.30 | 0.01 |
| RNA polymerase (beta subunit), rpoB | LMO0258 | Transcription | −0.47 | −0.76 | 0.02 |
| RNA polymerase (beta' subunit), rpoC | LMO0259 | Transcription | −0.12 | −0.46 | 0.00 |
| Ribosomal protein S6, rpsF | LMO0044 | Protein biosynthesis | −0.47 | −0.70 | 0.00 |
| Ribosomal protein S18, rpsR | LMO0046 | Protein biosynthesis | 0.15 | −0.67 | 0.00 |
| Ribosomal protein S21, rpsU | LMO1468 | Protein biosynthesis | 1.37 | −2.22 | 0.00 |
| Ribosomal protein L16, rplP | LMO2625 | Protein biosynthesis | −0.16 | −0.56 | 0.00 |
| Ribosomal protein L2, rplB | LMO2629 | Protein biosynthesis | −0.52 | −0.29 | 0.00 |
| Ribosomal protein S2, rpsB | LMO1658 | Protein biosynthesis | −0.26 | −1.04 | 0.02 |
| Ribosomal protein L27, rpmA | LMO1540 | Protein biosynthesis | −0.21 | −0.60 | 0.03 |
| Hypothetical ribosome-associated protein | LMO1541 | Protein biosynthesis | 0.00 | −0.59 | 0.03 |
| Ribosomal protein L15, rplO | LMO2613 | Protein biosynthesis | −0.62 | 0.06 | 0.23 |
| Ribosomal protein L23, rplW | LMO2630 | Protein biosynthesis | −0.38 | −0.19 | 0.29 |
| Translation elongation factor G | LMO2654 | Protein biosynthesis | −1.00 | −0.35 | 0.02 |
| Translation elongation factor EF-Tu | LMO2653 | Protein biosynthesis | −2.50 | −1.13 | 0.00 |
| Ribosomal-protein-alanine | LMO1301 | Protein biosynthesis | −0.18 | −0.40 | 0.23 |
| acetyltransferase | | | | | |
| Ribosome associated factor Y (global | LMO2511 | Protein biosynthesis | 1.41 | −3.85 | 0.00 |
| translation inhibitor) | | | | | |
| Acetyltransferase | LMO0624 | Post translational | 0.39 | −0.82 | 0.00 |
| | | modification | | | |
| ATP-dependent endopeptidase clp ATP- | LMO0997 | Protein degradation | −0.70 | −1.23 | 0.00 |
| binding subunit, clpE | | | | | |
| ATP-dependent endopeptidase clp ATP- | LMO1268 | Protein degradation | −0.78 | −0.41 | 0.17 |
| binding subunit, clpX | | | | | |
| ATP-dependent clp endopeptidase Clp | LMO2206 | Protein degradation | −0.72 | −0.07 | 0.10 |
| ATP-binding chain B, ClpB | | | | | |
| Oxidoreducatse involved in TATpathway | LMO0737 | Secretion | 0.61 | −2.64 | 0.00 |
| secreted proteins | | | | | |
| 60 kDa inner membrane protein yidC | LMO1379 | Secretion | −0.59 | −0.57 | 0.00 |
| Protein translocase subunit secY | LMO2612 | Secretion | −0.79 | −0.03 | 0.02 |
| 1-phosphatidylinositol phosphodiesterase | LMO0201 | Virulence | −0.33 | −0.92 | 0.00 |
| precursor, plcA | | | | | |
| listeriolysin O precursor, hly | LMO0202 | Virulence | 1.31 | −2.70 | 0.00 |
| Invasion associated protein, iap | LMO0582 | Virulence | −0.99 | −0.58 | 0.00 |
| Fibronectin-binding protein | LMO0721 | Virulence | 0.76 | −1.44 | 0.00 |
| General stress protein | LMO1601 | Stress | 0.16 | −0.73 | 0.00 |
| Universal stress protein family | LMO1580 | Stress | 0.66 | −0.19 | 0.00 |
| Toxic ion resistance proteins | LMO1967 | Stress | −0.74 | −0.29 | 0.00 |
| Arsenate reductase | LMO2230 | Stress | 0.59 | −1.03 | 0.00 |
| Superoxide dismutase, sod | LMO1439 | Stress | −1.39 | −4.35 | 0.00 |
| Non-heme iron-binding ferritin, fri | LMO0943 | Stress | 1.23 | −3.65 | 0.00 |
| Peptide methionine sulfoxide reductases, | LMO1860 | Stress | −1.68 | −0.25 | 0.00 |
| msrA | | | | | |
| Peptide methionine sulfoxide reductase, | LMO1859 | Stress | 0.19 | −0.90 | 0.00 |
| msrB | | | | | |
| Organic hydroperoxide resistance | LMO2199 | Stress | 0.79 | −1.51 | 0.00 |
| protein | | | | | |
| Thioredoxin reductase, trxB | LMO2478 | Stress | −1.61 | −0.63 | 0.00 |
| Heat shock protein, grpE | LMO1474 | Stress | −0.87 | −0.03 | 0.04 |
| Class I heat-shock protein (chaperonin), | LMO2068 | Stress | −0.88 | −0.44 | 0.03 |
| groEL | | | | | |
| Single-stranded DNA-binding protein, | LMO0045 | Stress | 0.53 | −3.84 | 0.00 |
| ssb | | | | | |
| Excinuclease ABC (subunit A), uvrA | LMO2488 | Stress | −0.53 | −0.84 | 0.04 |
| Endonuclease involved in recombination | LMO1502 | Stress/Cell division | −0.48 | −0.86 | 0.03 |
| DNA gyrase subunit B, gyrB | LMO0006 | Cell division | −0.85 | −0.29 | 0.00 |
| DNA gyrase subunit A, gyrA | LMO0007 | Cell division | −0.51 | −0.14 | 0.02 |
| ATPase associated with chromosome | LMO2759 | Cell division | 0.46 | −0.77 | 0.00 |
| architecture/replication | | | | | |
| Cell division initiation protein DivIVA | LMO1888 | Cell division | −0.65 | −0.28 | 0.03 |
| Hypothetical Protein | LMO0377 | Unknown | −0.08 | −0.73 | 0.00 |
| Hypothetical Protein | LMO0393 | Unknown | 0.87 | −0.95 | 0.00 |
| Hypothetical Protein | LMO0647 | Unknown | 1.00 | −2.42 | 0.00 |
| Hypothetical Protein | LMO1380 | Unknown | −0.63 | −0.07 | 0.00 |
| Hypothetical Protein | LMO1423 | Unknown | −1.04 | −1.28 | 0.00 |
| Hypothetical Protein | LMO1612 | Unknown | 1.20 | −1.69 | 0.00 |
| Hypothetical Protein | LMO2257 | Unknown | 0.74 | −0.75 | 0.00 |
| Hypothetical Protein | LMO2432 | Unknown | 3.52 | −3.23 | 0.00 |
| Hypothetical Protein | LMO2828 | Unknown | 0.79 | −1.05 | 0.00 |
| Hypothetical Protein | LMO2156 | Unknown | 0.72 | −0.88 | 0.01 |
| Hypothetical Protein | LMO1893 | Unknown | −0.06 | −0.51 | 0.03 |
| Hypothetical Protein | LMO1980 | Unknown | 0.58 | −0.39 | 0.07 |
| Hypothetical membrane spanning protein | LMO0625 | Unknown | 0.12 | −1.29 | 0.00 |
| Hypothetical membrane spanning protein | LMO0653 | Unknown | −0.52 | −0.31 | 0.01 |
| Hypothetical membrane associated | LMO2119 | Unknown | −0.62 | −0.54 | 0.00 |
| protein | | | | | |
| Hypothetical membrane spanning protein | LMO1690 | Unknown | −1.04 | −0.63 | 0.00 |
| GatB/Yqey domain protein | LMO1468 | Unknown | 0.25 | −0.92 | 0.00 |
| Hypothetical cytosolic protein | LMO1501 | Unknown | 0.05 | −1.45 | 0.00 |
| Hypothetical cytosolic protein | LMO2472 | Unknown | −0.58 | 0.21 | 0.00 |
| Hypothetical cytosolic protein | LMO0964 | Unknown | −3.18 | −0.84 | 0.00 |
| Cytosolic protein containing multiple | LMO1576 | Unknown | −1.18 | −0.16 | 0.00 |
| CBS domains | | | | | |
| Stage V sporulation protein G | LMO0197 | Others | −0.38 | −0.31 | 0.23 |
| Stage V sporulation protein G | LMO0196 | Others | −0.72 | −0.27 | 0.23 |
| Acetyl esterase | LMO2089 | Others | −0.30 | −1.19 | 0.00 |
| Protease I | LMO2256 | Others | 0.83 | −2.28 | 0.00 |
| Glyoxalase family protein | LMO2437 | Others | 2.91 | −4.22 | 0.00 |
| Predicted hydrolases or acyltransferases | LMO2453 | Others | −0.80 | 0.13 | 0.10 |
| (alpha/beta hydrolase superfamily) | | | | | |
| Hydrolase (HAD superfamily) | LMO1399 | Others | −0.87 | −0.38 | 0.17 |
| Putative transcriptional regulator, AraC | LMO0109 | Others | −0.50 | −0.14 | 0.10 |
| family | | | | | |
| Putative ranscriptional regulator, ArsR | LMO0101 | Others | −0.30 | 0.58 | 0.03 |
| family | | | | | |
| Putative transcription regulator | LMO0740 | Others | 0.13 | 0.76 | 0.29 |
| Phosphoesterase, DHH family protein | LMO1575 | Others | −0.56 | −0.37 | 0.00 |
| Carboxylesterase | LMO2452 | Others | −0.92 | 0.03 | 0.00 |
| Glycerol uptake facilitator protein | LMO1539 | Others | −0.56 | −0.37 | 0.07 |
| Permease | LMO2148 | Others | 0.15 | 0.64 | 0.17 |

TABLE 7
Table 7. Therapeutic solutions of RLs and SP 25A.

| | |
|---|---|
| Component | % (by weight) |

FORMULATION 2A

| | |
|---|---|
| Syringopeptin 25A | 5 |
| Rhamnolipid | 6 |
| Citricidal ™ (Bio/Chem Research, Petaluma, CA) | 0.5 |
| Water | 87.5 |
| 1% saline solution | 1 |

FORMULATION 3A

| | |
|---|---|
| Syringopeptin 25A | 6 |
| Rhamnolipid | 5 |
| Citricidal ™ (Bio/Chem Research, Petaluma, CA) | 0.5 |
| Purified water | 88.5 |

FORMULATION 4A

| | |
|---|---|
| Syringopeptin 25A | 10 |
| Rhamnolipid | 11 |
| Citricidal ™ (Bio/Chem Research, Petaluma, CA) | 0.5 |
| Phosphoric acid | 1 |
| Sodium bicarbonate | 1.5 |
| Purified water | 77 |

FORMULATION 5A

| | |
|---|---|
| Syringopeptin 25A | 5 |
| Rhamnolipid | 15 |
| Citricidal ™ (Bio/Chem Research, Petaluma, CA) | 0.5 |
| 1% saline solution | 79.5 |

FORMULATION 6A

| | |
|---|---|
| Syringopeptin 25A | 8 |
| Rhamnolipid | 2 |
| Carboxymethylcellulose (15,000 Mw) | 0.5 |
| Castor oil | 0.5 |
| 1% saline solution | 89 |

FORMULATION 7A

| | |
|---|---|
| Syringopeptin 25A | 5 |
| Rhamnolipid | 5 |
| Xylitol | 5 |
| Carboxymethylcellulose (15,000 Mw) | 0.5 |
| Citricidal ™ (Bio/Chem Research, Petaluma, CA) | 0.5 |
| 1% saline solution | 84 |

FORMULATION 8A

| | |
|---|---|
| Syringopeptin 25A | 9 |
| Rhamnolipid | 2 |
| Xylitol | 10 |
| Citricidal ™ (Bio/Chem Research, Petaluma, CA) | 0.5 |
| 1% saline solution | 78.5 |

TABLE 8
Table 8. Therapeutic compositions of RLs and SP 25A.

| | |
|---|---|
| Component | % (by weight) |

FORMULATION 2B

| | |
|---|---|
| Syringopeptin 25A | 12.5 |
| Rhamnolipid | 12.5 |
| Carboxymethylcellulose (15,000 Mw) | 2 |
| Calcium glycerol phosphate | 6.5 |
| Methyl laurate | 5 |
| Menthol | 1 |
| Purified water | 60.5 |

FORMULATION 3B

| | |
|---|---|
| Syringopeptin 25A | 12.5 |
| Rhamnolipid | 12.5 |
| Xylitol | 5 |
| Carboxymethylcellulose (15,000 Mw) | 12 |
| Calcium glycerol phosphate | 2.5 |
| Lauryl alcohol | 5 |
| Propylene glycol | 5 |
| Purified Water | 45.5 |

FORMULATION 4B

| | |
|---|---|
| Syringopeptin 25A | 15 |
| Rhamnolipid | 20 |
| Carboxymethylcellulose (15,000 Mw) | 21 |
| Calcium glycerol phosphate | 6.5 |
| Phosphoric acid | 1 |
| Sodium bicarbonate | 1.5 |
| Purified Water | 35 |

FORMULATION 5B

| | |
|---|---|
| Syringopeptin 25A | 10 |
| Rhamnolipid | 10 |
| Erythritol | 20 |
| Carboxymethylcellulose (15,000 Mw) | 25 |
| Calcium glycerol phosphate | 4 |
| Calcium phosphate | 1 |
| Purified Water | 30 |

FORMULATION 6B

| | |
|---|---|
| Syringopeptin 25A | 10 |
| Rhamnolipid | 10 |
| Erythritol | 25 |
| Carboxymethylcellulose (15,000 Mw) | 20 |
| Gum arabic | 1 |
| Calcium glycerol phosphate | 5 |
| Castor oil | 1 |
| Purified Water | 28 |

FORMULATION 7B

| | |
|---|---|
| Syringopeptin 25A | 5 |
| Rhamnolipid | 5 |
| Erythritol | 40 |
| Carboxymethylcellulose (15,000 Mw) | 20 |
| Calcium glycerol phosphate | 5 |
| Cyclodextran | 1 |
| Purified Water | 24 |

FORMULATION 8B

| | |
|---|---|
| Syringopeptin 25A | 2.5 |
| Rhamnolipid | 2.5 |
| Xylitol | 25 |
| Erythritol | 25 |
| Carboxymethylcellulose (15,000 Mw) | 15.5 |
| Calcium glycerol phosphate | 5 |
| Menthol | 0.5 |
| Purified Water | 24 |

FORMULATION 9B

| | |
|---|---|
| Syringopeptin 25A | 1 |
| Rhamnolipid | 1 |
| Erythritol | 33 |
| Ticalose ® (TIC Gums, Belcamp, MD) | 2.5 |
| Calcium | 1.5 |
| Grapefruit seed extract | <0.1 |
| Purified Water | ~61 |

TABLE 9
Table 9. The effects of SP25A on selected human cancer cell lines as % cytotoxicity.
Cancer Type (cell line name)

| Conc. | Lung | Stomach | Prostate | Cervical | Colon | Breast | | | Ovarian | Bone |
|---|---|---|---|---|---|---|---|---|---|---|
| (ug/ml) | (A549) | (AGS) | (DU145) | (HeLa) | (HT29) | (MDA-MB231) | (MDA-MB435) | (MCF-7) | (OVCAR3) | (U2OS) |
| 10.00 | 43.82 | 26.90 | 76.25 | 19.61 | 16.83 | 49.30 | 79.98 | 16.52 | 25.31 | 4.93 |
| 5.00 | 41.14 | 26.88 | 45.22 | 1.08 | 24.59 | 21.98 | 18.84 | 1.76 | 16.41 | 1.69 |
| 2.50 | 13.51 | 13.87 | 23.60 | 3.50 | 23.85 | 9.23 | −5.22 | −1.12 | 16.30 | −3.64 |
| 1.00 | 15.66 | 4.00 | 26.92 | 1.68 | 8.89 | −12.40 | 20.78 | −20.40 | 6.42 | −10.06 |
| IC50 | 11.41 | 18.58 | 6.56 | 25.50 | 29.72 | 10.14 | 6.25 | 30.27 | 19.75 | 101.47 |

What is claimed is:

1. A therapeutic composition comprising:
a therapeutically effective amount of a syringopeptin;
a therapeutically effective amount of a rhamnolipid; and
a pharmaceutically acceptable carrier.

2. A therapeutic composition as in claim 1, wherein the syringopeptin has an amino acid backbone of 22 or 25 amino acids.

3. A therapeutic composition as in claim 1, wherein the syringopeptin has a polypeptide sequence as in SEQ ID No. 1 or 2.

4. A therapeutic composition as in claim 1, wherein the syringopeptin has a polypeptide sequence with at least 90% homology with SEQ ID No. 1 or 2.

5. A therapeutic composition as in claim 1, wherein a N-terminal amino acid residue of the syringopeptin is acylated by a 3-hydroxylated fatty acid chain comprising 10 or 12 carbon atoms.

6. A therapeutic composition as in claim 1, wherein a C-terminal amino acid carboxyl group of the syringopeptin is linked to an amino acid residue 7 residues away and form an 8-membered lactone macrocycle.

7. A therapeutic composition as in claim 1, wherein the rhamnolipid has the following structure: 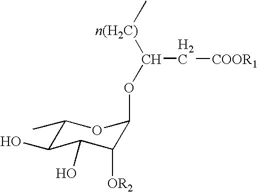
wherein n is from 4-12;
R₁ is H or 3-hydroxydecanoate; and
R₂ is L-rhamnosyl or H.

8. A therapeutic composition as in claim 1, wherein the ratio of syringopeptin and rhamnolipid ranges from about 1:10 to about 10:1.

9. A therapeutic composition as in claim 1, wherein the therapeutically effective amounts of syringopeptin and rhamnolipid achieve a minimum inhibitory concentration in a subject sufficient to prevent, alleviate, or eliminate a microbial infection.

10. A therapeutic composition as in claim 9, wherein the microbial infection is tuberculosis.

11. A therapeutic composition as in claim 1, wherein the therapeutically effective amounts of syringopeptin and rhamnolipid achieve a minimum inhibitory concentration in a subject sufficient to prevent tumor formation, reduce tumor growth, reduce tumor size, or kill tumor cells.

12. A method for inhibiting or treating cancer in a subject, the method comprising:
providing a subject in need of inhibition or treatment of cancer; and
administering a therapeutic amount of a therapeutic composition to the subject so as to inhibit or treat the cancer, the therapeutic composition comprising:
a therapeutically effective amount of a syringopeptin;
a therapeutically effective amount of a rhamnolipid; and
a pharmaceutically acceptable carrier.

13. A method as in claim 12, wherein the cancer may be in the form of a benign or malignant tumor.

14. A method as in claim 12, wherein the subject is a human.

15. A method as in claim 12, wherein the syringopeptin is characterized by at least one of the following:
the syringopeptin has a polypeptide sequence as in SEQ ID No. 1 or 2;
a N-terminal amino acid residue of the syringopeptin is acylated by a 3-hydroxylated fatty acid chain comprising 10 or 12 carbon atoms; or
a C-terminal amino acid carboxyl group of the syringopeptin is linked to an amino acid residue 7 residues away to form an 8-membered lactone macrocycle.

16. A method as in claim 12, wherein the rhamnolipid has the following structure: 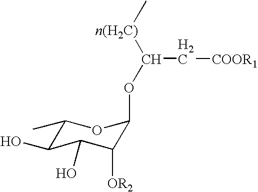
wherein n is from 4-12;
R₁ is H or 3-hydroxydecanoate; and
R₂ is L-rhamnosyl or H.

17. A method as in claim 12, wherein the ratio of syringopeptin and rhamnolipid ranges from about 1:10 to about 10:1.

18. A method for inhibiting or treating a microbial infection in a subject, the method comprising:
providing a subject in need of inhibition or treatment for a microbial infection; and
administering a therapeutic amount of a therapeutic composition to the subject so as to inhibit or treat the microbial infection, the therapeutic composition comprising:
a therapeutically effective amount of a syringopeptin;
a therapeutically effective amount of a rhamnolipid; and
a pharmaceutically acceptable carrier.

19. A method as in claim 18, wherein the infection may be in the form of a localized or systemic infection.

20. A method as in claim 18, wherein the microbial infection is caused by Mycobacterium tuberculosis.

21. A method as in claim 18, wherein the subject is a human.

22. A method as in claim 18, wherein the syringopeptin is characterized by at least one of the following:
the syringopeptin has a polypeptide sequence as in SEQ ID No. 1 or 2;
a N-terminal amino acid residue of the syringopeptin is acylated by a 3-hydroxylated fatty acid chain comprising 10 or 12 carbon atoms; or
a C-terminal amino acid carboxyl group of the syringopeptin is linked to an amino acid residue 7 residues away to form an 8-membered lactone macrocycle.

23. A method as in claim 18, wherein the rhamnolipid has the following structure: 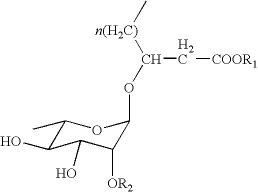
wherein n is from 4-12;
R₁ is H or 3-hydroxydecanoate; and
R₂ is L-rhamnosyl or H.

24. A method as in claim 18, wherein the ratio of syringopeptin and rhamnolipid ranges from about 1:10 to about 10:1.

25. A therapeutic composition for use in treating and/or preventing an illness in a subject, the therapeutic composition comprising:
a pharmaceutically acceptable carrier;
a syringopeptin having a polypeptide sequence as in SEQ ID No. 1 or 2 at a concentration of at least about 3 μg/mL within the carrier; and
a rhamnolipid at a concentration of at least about 3 μg/mL, wherein the rhamnolipid has a structure as in Structure 1 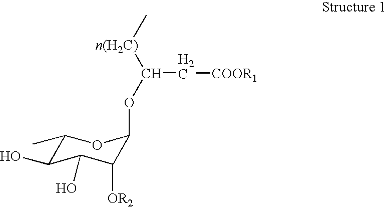
wherein, n is from 4-12;
R₁ is H or 3-hydroxydecanoate; and
R₂ is L-rhamnosyl or H.

26. A therapeutic composition as in claim 25, wherein a N-terminal amino acid residue of the syringopeptin is acylated by a 3-hydroxylated fatty acid chain comprising 10 or 12 carbon atoms.

27. A therapeutic composition as in claim 25, wherein a C-terminal amino acid carboxyl group of the syringopeptin is linked to an amino acid residue 7 residues away and form an 8-membered lactone macrocycle.

28. A therapeutic composition as in claim 25, wherein the ratio of syringopeptin and rhamnolipid ranges from about 1:10 to about 10:1.

29. A therapeutic composition as in claim 25, wherein the syringopeptin and rhamnolipid are present in the carrier in an amount sufficient to achieve a minimum inhibitory concentration in a subject sufficient to treat and/or prevent the illness in the subject.

30. A therapeutic composition as in claim 29, wherein the illness is a microbial infection.

31. A therapeutic composition as in claim 30, wherein the microbial infection is tuberculosis.

32. A therapeutic composition as in claim 25, wherein the syringopeptin and rhamnolipid are present in the carrier in an amount sufficient to achieve a minimum inhibitory concentration in a subject sufficient to prevent tumor formation, reduce tumor growth, reduce tumor size, or kill tumor cells.

33. A therapeutic composition as in claim 25, wherein the composition is in the form of a tablet, pill, capsule, semisolid, powder, sustained release formulation, solution, suspension, elixir, aerosol, gel cap, caplet, suppositorie, or combination thereof.

34. A therapeutic composition as in claim 25, wherein the carrier is configured to be administered to the subject by a route selected from the group consisting of orally, systemically, transdermally, intranasal, suppository, parenteral, intramuscular, intravenous, subcutaneous, injection, implantation, vaginally, rectally, buccally, pulmonary, topically, nasally, and combination thereof.

35. A therapeutic composition as in claim 25, wherein the carrier is selected from the group consisting of ion exchangers, alumina, aluminum stearate, lecithin, serum proteins, human serum albumin, buffers, phosphates, glycine, sorbic acid, potassium sorbate, partial glyceride mixtures of saturated vegetable fatty acids, water, salts, electrolytes, prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and combinations thereof.

36. A therapeutic composition as in claim 25, further comprising a pharmaceutically acceptable excipient.

37. A therapeutic composition as in claim 36, wherein the excipients is selected from the group consisting of acidulents, lactic acid, hydrochloric acid, tartaric acid, solubilizing components, non-ionic surfactant, cationic surfactant, anionic surfactant, absorbents, bentonite, cellulose, kaolin, alkalizing components, diethanolamine, potassium citrate, sodium bicarbonate, anticaking components, calcium phosphate tribasic, magnesium trisilicate, talc, antioxidants, ascorbic acid, alpha tocopherol, propyl gallate, sodium metabisulfite, binders, acacia, alginic acid, carboxymethyl cellulose, hydroxyethyl cellulose, dextrin, gelatin, guar gum, magnesium aluminum silicate, maltodextrin, povidone, starch, vegetable oil, buffering components, sodium phosphate, malic acid, potassium citrate, chelating components, EDTA, malic acid, maltol, coating components, sugar, cetyl alcohol, polyvinyl alcohol, carnauba wax, lactose maltitol, titanium dioxide, microcrystalline wax, white wax, yellow wax, desiccants, calcium sulfate, detergents, lauryl sulfate, diluents, calcium phosphate, sorbitol, starch, lactitol, polymethacrylates, sodium chloride, glyceryl palmitostearate, disintegrants, colloidal silicon dioxide, croscarmellose sodium, magnesium aluminum silicate, potassium polacrilin, sodium starch glycolate, dispersing components, poloxamer 386, polyoxyethylene fatty esters, polysorbates, emollients, cetearyl alcohol, lanolin, mineral oil, petrolatum, cholesterol, isopropyl myristate, lecithin, emulsifying components, anionic emulsifying wax, monoethanolamine, medium chain triglycerides.

38. A therapeutic composition as in claim 25, further comprising a flavoring component selected from the group consisting of ethyl maltol, ethyl vanillin, fumaric acid, malic acid, maltol, and menthol.

39. A therapeutic composition as in claim 25, further comprising humectant selected from the group consisting of glycerin, propylene glycol, sorbitol, and triacetin.

40. A therapeutic composition as in claim 25, further comprising a lubricant selected from the group consisting of calcium stearate, canola oil, glyceryl palmitostearate, magnesium oxide, poloxymer, sodium benzoate, stearic acid, and zinc stearate.

41. A therapeutic composition as in claim 25, further comprising a solvent selected from the group consisting of alcohols, benzyl phenylformate, vegetable oils, diethyl phthalate, ethyl oleate, glycerol, glycofurol, and polyethylene glycol.

42. A therapeutic composition as in claim 25, further comprising a stabilizing component selected from the group consisting of cyclodextrins, albumin, polysaccharides, starch, cellulose, xanthan gum and combinations thereof.

43. A therapeutic composition as in claim 25, further comprising a tonicity component selected from the group consisting of glycerol, dextrose, potassium chloride, sodium chloride, and combinations thereof.

44. A therapeutic composition as in claim 25, further comprising an antimicrobial component selected from the group consisting of benzoic acid, sorbic acid, benzyl alcohol, benzethonium chloride, bronopol, alkyl parabens, cetrimide, phenol, phenylmercuric acetate, thimerosol, phenoxyethanol, and combinations thereof.

45. A therapeutic composition as in claim 25, further comprising a pharmaceutical agent selected from the group consisting of antibiotics, anti-parasitic agents, antifungal agents, anti-viral agents, and anti-tumor agents.


SEQUENCE LISTING (2 sequences)

SEQ ID NO: 1 - protein, 22 aa, Artificial Sequence
  Description of Artificial Sequence Synthetic polypeptide
XPVVAAVVXAVAAXXSAXAXXY

SEQ ID NO: 2 - protein, 25 aa, Artificial Sequence
  Description of Artificial Sequence Synthetic polypeptide
XPVAAVLAAXVXAVAAXXSAVAXXY